(19) United States
(12) Patent Application Publication
(10) Pub. No.: US 2020/0123487 A1
(43) Pub. Date: Apr. 23, 2020

(54) MICROFLUIDIC PLATELET BIOREACTOR DEVICE AND SYSTEMS

(71) Applicant: Northwestern University, Evanston, IL (US)

(72) Inventors: William M. Miller, Evanston, IL (US); Richard McMahon, Evanston, IL (US); Andres Martinez, Evanston, IL (US)

(21) Appl. No.: 16/624,681

(22) PCT Filed: Jun. 20, 2018
(86) PCT No.: PCT/US2018/038579

§ 371 Date: Dec. 19, 2019

Related U.S. Application Data
(60) Provisional application No. 62/642,955, filed on Mar. 14, 2018.
(60) Provisional application No. 62/522,491, filed on Jun. 20, 2017.

(51) Int. Cl. C12M 3/06; C12M 1/00; B01J 19/00; C12N 5/078

(52) U.S. Cl. CPC C12M 23/16; C12M 21/00; B01J 2219/00905; C12N 5/0644; B01J 2219/00903; B01J 19/0093

(57) ABSTRACT

The present disclosure relates to the production of platelets from megakaryocytes (Mks). In particular, the present disclosure provides systems and methods for the in vitro production of platelets from Mks using a microfluidic bioreactor having a center flow channel and uniform high-shear micrometer slits. Use of this microfluidic bioreactor enables the continuous production of millions of platelets and facilitates real-time and long-term visualization of proplatelet and platelet generation.

### RELATED APPLICATIONS

This application claims priority to and the benefit of U.S. Provisional Patent Application No. 62/522,491 filed Jun. 20, 2017, and U.S. Provisional Patent Application No. 62/642,955 filed Mar. 14, 2018, both of which are incorporated herein by reference in their entireties for all purposes.

### GOVERNMENT FUNDING

This subject matter of this invention was made with Government support under Grant No. 5R01HL130760-03 awarded by the National Institutes of Health (NHLBI), and Grant No. CBET1265029 awarded by the National Science Foundation (NSF). The Government has certain rights in the invention.

### FIELD

The present disclosure relates to the production of platelets from megakaryocytes (Mks). In particular, the present disclosure provides systems and methods for the in vitro production of platelets from Mks using a microfluidic bioreactor having a center flow channel and uniform high-shear micrometer slits. Use of this microfluidic bioreactor enables the continuous production of millions of platelets and facilitates real-time and long-term visualization of proplatelet and platelet generation.

### BACKGROUND

More than 2 million platelet units are transfused each year in the U.S. to treat patients with thrombocytopenia (low platelet counts) or defective platelets. Platelets are small (2-3 μm) anucleate discoids responsible for thrombosis and hemostasis. Platelet units are collected from volunteer donors via apheresis or from the huffy coats of 4-8 whole blood donations. Hospitals depend on a steady supply of platelet donors. Disruptions of this supply together with a 5-day platelet shelf-life can result in critical shortages. In addition, because platelets require room temperature storage to maintain activity, there is risk of bacterial contamination prior to transfusion. The use of a current Good Manufacturing Practices (cGMP) process for platelet production from megakaryocytes (Mks) would allow for better control and characterization of transfusion units, and could transition the supply from fluctuating donors to a steady in vitro process. Finally, culture-derived platelet production could reduce the risk of immunogenic reactions by avoiding the need to provide platelets from multiple donors.

Platelet formation starts from Mks, which undergo extensive cytoskeletal rearrangements to create proplatelets (proPLTs), the precursors to platelets. In the bone marrow, intravital microscopy studies in mice show that Mks directionally extend proPLTs into the blood sinuses, where shear forces (1.3-4.1 dynes/cm²) elongate and fragment proPLTs into preplatelets (prePLTs). High shear stress in the lung capillary bed shears proPLTS and prePLTs into individual platelets, and can also process Mks directly into platelets. The importance of shear forces has led to the experimental use of bioreactors to study and enhance platelet-like-particle (PLP) release from mature Mks. Estimates of platelet production in vivo are typically greater than 1000 PLPs per Mk; in comparison, in vitro studies typically report less than 100 PLPs per Mk, and in many cases, less than 10 PLPs per Mk.

A major challenge in the development of platelet bioreactors is that much remains unknown about the ex vivo initiation and regulation of proPLT formation, as well as how to maximize PUP release. Parallel-plate flow reactors (PPFRs) are the simplest bioreactors that have been used to study proPLT/PLP formation from adhered Mks under high (1800 s⁻¹)13 and low (400 s⁻¹) shear rates. However, it is difficult to carry out long-term analysis of individual Mks due to transient adhesions as Mks roll over the PPFR surface. Improvements to open-channel PPFRs include introducing an array of vWF (Von Willebrand factor)-coated columns in bioreactors. The anchoring of Mks to columns, at a shear rate of 5000 s⁻¹, allowed longer Mk retention for analysis and study of the proPLT formation step. Complex niche bioreactors occupy the other end of the bioreactor spectrum. For example, a 3D silk-based porous microtube surrounded by a silk sponge reproduced the structure of a blood sinus and the bone marrow niche. Using a shear rate of 60 s⁻¹, the system reproduced PLP production in a physiologically relevant environment, but real-time visualization was challenging. Therefore, insight into the factors that regulate proPLT formation and PLP release could be limited since immediate changes to the proPLT formation process cannot be analyzed. Similar limitations in real-time visualizations are present in a porous membrane system through which Mks extended proPLTs into a lower chamber with shear rates of 30-70 s⁻¹.

In contrast, slit bioreactors, which use small features to create <10-μm openings that mimic gaps or fenestrations in endothelial cells lining sinuses in the bone marrow, offer the advantage of in situ study and analysis of proPLT and PLP formation that is difficult in the other types of bioreactors. Some such bioreactors use a 4-μm slit bioreactor with unspecified shear rates and others include a 2-μm slit bioreactor with a shear rate of 500 s⁻¹. Although the PDMS-based fabrication of these slit bioreactors facilitate visualization of the proPLT formation process in real-time, the flow patterns and shear rates within current systems have not been identified. Developing an understanding of the bioreactor flow environment is important since non-uniformity in the flow patterns would lead to Mks experiencing different shear rates depending on slit location.

### SUMMARY

Embodiments of the present disclosure include a microfluidic proplatelet (proPLT) and platelet-like particle (PLP) production chamber device. In accordance with these embodiments, the device includes a plurality of slit channels that include one or more proPLT/PLP production slits configured to expose a megakaryocyte to a uniform shear profile to facilitate the production of proPLTs/PLPs, with the plurality of slit channels distal to and in fluid communication with a central flow channel.

In some embodiments, the one or more proPLT/PLP production slits are from about 3 μm to about 10 μm wide. In some embodiments, the one or more proPLT/PLP production slits are from about 5 μm to about 7 μm wide. In some embodiments, the one or more proPLT/PLP production slits are about 5 μm wide.

In some embodiments, the uniform shear profile produces a maximum shear rate at the one or more proPLT/PLP production slits ranging from about 5000 s⁻¹ to about 10,000 s⁻¹. In some embodiments, the uniform shear profile produces a maximum shear rate at the one or more proPLT/PLP production slits ranging from about 5000 s⁻¹ to about 8,000 s⁻¹. In some embodiments, the uniform shear profile produces a maximum shear rate at the one or more proPLT/PLP production slits of about 5000 s⁻¹, and the one or more proPLT/PLP production slits are about 7 μm wide. In some embodiments, the uniform shear profile produces a maximum shear rate at the one or more proPLT/PLP production slits of about 8,200 s⁻¹, and the one or more proPLT/PLP production slits are about 5 μm wide.

In some embodiments, the uniform shear profile produces a shear rate on the proPLTs/PLPs ranging from about 50 s⁻¹ to about 500 s⁻¹. In some embodiments, the uniform shear profile produces a shear rate on the proPLTs/PLPs ranging from about 250 s⁻¹ to about 350 s⁻¹. In some embodiments, the uniform shear profile produces a shear rate on the proPLTs/PLPs ranging from about 50 s⁻¹ to about 100 s⁻¹. In some embodiments, the uniform shear profile produces a shear rate on the proPLTs/PLPs ranging from about 250 s⁻¹ to about 350 s⁻¹, and the one or more proPLT/PLP production slits are about 7 μm wide. In some embodiments, the uniform shear profile produces a shear rate on the proPLTs/PLPs ranging from about 50 s⁻¹ to about 100 s⁻¹, and the one or more proPLT/PLP production slits are about 5 μm wide.

In some embodiments, fluid flows in a single direction from the central flow channel to the plurality of slit channels. In some embodiments, the single direction fluid flow produces at least a 5-fold increase in proPLT/PLP production. In some embodiments, the device further includes a proPLT/PLP collection reservoir.

Embodiments of the present disclosure include a microfluidic bioreactor device for the production of proplatelets (proPLTs) and platelet-like particles (PLPs). In accordance with these embodiments, the device includes a megakaryocyte loading reservoir coupled to a central flow channel, a branching flow channel in fluid communication with the central flow channel, and a proPLT/PLP production chamber that includes a plurality of slit channels distal to and in fluid communication with the branching flow channel, with the plurality of slit channels having one or more proPLT/PLP production slits configured to expose a megakaryocyte to a uniform shear profile to facilitate the production of proPLTs/PLPs.

In some embodiments, the one or more proPLT/PLP production slits are from about 5 μm to about 7 μm wide. In some embodiments, the uniform shear profile produces a maximum shear rate at the one or more proPLT/PLP production slits of about 5000 s⁻¹, and the one or more proPLT/PLP production slits are about 7 μm wide. In some embodiments, the uniform shear profile produces a maximum shear rate at the one or more proPLT/PLP production slits of about 8,200 s⁻¹, and the one or more proPLT/PLP production slits are about 5 μm wide. In some embodiments, the uniform shear profile produces a shear rate on the proPLTs/PLPs ranging from about 250 s⁻¹ to about 350 s¹, and the one or more proPLT/PLP production slits are about 7 μm wide. In some embodiments, the uniform shear profile produces a shear rate on the proPLTs/PLPs ranging from about 50 s⁻¹ to about 100 s⁻¹, and the one or more proPLT/PLP production slits are about 5 μm wide. In some embodiments, fluid flows in a single direction from the central flow channel to the plurality of slit channels.

Embodiments of the present disclosure include a microfluidic bioreactor system for the production of proplatelets (proPLTs) and platelet-like particles (PLPs). In accordance with these embodiments, the system includes a megakaryocyte loading reservoir coupled to a central flow channel, a branching flow channel in fluid communication with the central flow channel, and a proPLT/PLP production chamber having a plurality of slit channels distal to and in fluid communication with the branching flow channel, with the plurality of slit channels having one or more proPLT/PLP production slits configured to expose a megakaryocyte to a uniform shear profile to facilitate the production of proPLTs/PLPs.

In some embodiments, the system further includes a proPLT/PLP collection reservoir. In some embodiments, the system further includes a fluid source coupled to the central flow channel and configured to supply fluid pressure to the system.

### BRIEF DESCRIPTION OF THE DRAWINGS

FIGS. 1A-1B include representative schematic diagrams depicting shear rate ranges that proPLTs are subject to within published bioreactor systems and embodiments of the present disclosure (FIG. 1A), including maximum shear rates that Mk bodies are subject to within published cell-free bioreactor systems (FIG. 1B). These diagrams show the wide spectrum of shear rates that have been studied in bioreactors, as well as in vivo values for bone marrow sinusoids and the lung.

FIGS. 2A-2B include representative schematic diagrams depicting computational fluid dynamics (CFD) of published bioreactors. FIG. 2A: shear profile of Nakagawa et al. system; inlet flow rates=16.7 μL/min. FIG. 2B: shear rates in the Thon et al. system; inlet flow rate for each channel was set to 6.25 μL/h (total combined inlet flow rate 12.5 μL/h). White arrows indicate the flow direction. Insets of both systems are shown for details around the slit channels that Mks occupy (insets rotated to show all the channel walls).

FIGS. 3A-3C include representative schematic diagrams of the design concept of the uniform-shear-rate bioreactor and experimental set up, according to one embodiment of the present disclosure. FIG. 3A: the present system can include an array of 7-μm slits to capture Mks. Outside flows converge at the slits to apply shear forces on extending proPLTs. FIG. 3B: two syringe pumps can be used for the bioreactor operation, allowing for independent flow rate changes to the center and outer channels. The bioreactor is positioned over a microscope equipped with real-time imaging in brightfield and green fluorescence. The entire system is placed inside an incubator at 37° C., 20% O₂, and 5% CO₂. FIG. 3C: cell-free fabricated bioreactor. Scale bar=50 μm.

FIGS. 4A-4B include representative results of qualitative validation of the bioreactor flow profiles, according to one embodiment of the present disclosure. FIG. 4A: comparison of streamline plots from CFD simulations. FIG. 4B: experimental flow visualizations using 1-μm fluorescent beads. The flow rate is 1 μL/min in the center channel and in the combined outer channels. White arrows indicate the flow direction. Scale bar=50 μm.

FIGS. 5A-5D include representative results of shear rate analysis of a cell-free bioreactor, according to one embodiment of the present disclosure. FIG. 5A: shear rates in the entire slit region. FIG. 5B: close-up view of individual slits for 1.5 μL/min flow rates in the center and combined outer channels. FIG. 5C: shear rates through the entire region. FIG. 5D: details for individual slits for a flow rate of 1.5 μL/min in the center channel with 5 μL/min in the combined outer channels. White arrows indicate the general flow direction. Estimated shear rates on proPLTs (dashed lines) are within 100 μm from the slits.

FIGS. 6A-6B include representative results of shear analysis using 20-μm spheres near the slits (2-μm gap size upstream of posts), according to one embodiment of the present disclosure. Center of spheres are placed at the center of the bioreactor height (z=20 μm). FIG. 6A: shear rates through the entire region. FIG. 6B: details for individual slits for a flow rate of 1.5 μL/min in the center channel with 5 μL/min in the combined outer channels. White arrows indicate the flow direction. Estimated shear rates on cells designated by (*). Estimated shear rates on proPLTs (dashed lines) are within 100 μm from the slits.

FIGS. 7A-7C include representative results of the modeling of cell blockage of bioreactor slits, according to one embodiment of the present disclosure. FIG. 7A: shear rates after blocking all but 2 of the slits. FIG. 7B: velocity streamlines of a system with 2 open slits, FIG. 7C: visualization of the system dynamics with fluorescent beads and Calcein-stained cells. Simulation and experimental flow rates were 1.5 μL/min in the center channel with a combined flow of 5 μL/min in the outer channels. White arrows indicate the flow direction. Estimated shear rates on proPLTs are within 100 μm from the slits. Scale bar=50 μm.

FIGS. 8A-8B include representative results of shear-driven proPLT formation in the bioreactor, according to one embodiment of the present disclosure. FIG. 8A: green fluorescence observation of Calcein-labeled Mks with proPLT formation. FIG. 8B: brightfield time-lapse images of cells trapped in a slit and exposed to shear. The center channel flow elongates Mks through the slits. Flow in the outer channels applies shear on the extensions further elongating them leading to fragmentation after several minutes. Black arrows indicate proPLTs. Scale bar=50 μm. Flow rate of 1.5 μL/min in the center channel with 5 μL/min in the combined outer channels. Blue arrows show direction of flow.

FIG. 9 includes representative results of shear-driven rapid PLP release in the bioreactor, according to one embodiment of the present disclosure. Time-lapse images of trapped Mks in a slit, rapidly releasing many individual PLPs in seconds. Time units: h:min:s. Orange arrows point to individual PLPs. Scale bar=50 μm. Blue arrows show direction of flow. Flow rate of 1.5 μL/min in the center channel with 5 μL/min in the combined outer channels.

FIGS. 10A-10B include representative results of PLP release kinetics in the bioreactor, according to one embodiment of the present disclosure. FIG. 10A: number of PLPs released per 5-min time interval during a bioreactor run with constant center channel flow rate (indicated above plot) and three significant cell blockages of open slits during that time interval denoted by green arrows. FIG. 10B: number of PLPs released per 5-min time interval during a bioreactor run with three center channel flow rate increases (dashed lines—indicated above plot) and three significant cell blockages of open slits denoted by green arrow. Combined outer channels flow rate=5 μL/min. Color legend depicts the number of slits making PLPs during each 5-min time interval.

FIGS. 11A-11B include representative results of PLP release kinetics in the bioreactor with no outer flow channel, according to one embodiment of the present disclosure. FIG. 11A: number of PLPs released per 5-min time interval during a bioreactor run with no center channel flow rate changes (indicated above plot) and four significant cell blockages of open slits denoted by green arrow. FIG. 11B: number of PLPs released per 5-min time interval during a bioreactor run with two center channel flow rate changes (dashed lines—indicated above plot) and three significant cell blockages of open slits denoted by green arrow. Combined outer channels flow rate=0 μL/min. Color legend depicts the number of slits making PLPs during each 5-min time interval.

FIGS. 12A-12C include representative results of the distributions of PLP release kinetics in the bioreactor, according to one embodiment of the present disclosure. FIG. 12A: number of slits open per 5-min time interval for (i) 5 μL/min and (ii) 0 μL/min combined outside channel flow rate. FIG. 12B: % occupied slits that were making PLPs per 5-min time interval for (i) 5 μL/min and (ii) 0 μL/min outside flow rate. FIG. 12C: PLPs released per 5-min time interval for (i) 5 μL/min (exponential fit) and (ii) 0 μL/min (log-normal fit) outside flow rate. Error bars indicate±SEM.

FIGS. 13A-13C include representative results of the characterization of recovered PLPs from effluent, according to one embodiment of the present disclosure. FIG. 13A: CD41 and CD42b expression of Calcein⁺ PLPs. FIG. 13B: representative plots of activation of recovered CD41⁺CD42b⁺ PLPs in the absence (i) or presence (ii) of thrombin and (iii) summary of % CD62P⁺ PLPs for 3 bioreactor experiments from two Mk cultures with different donors. FIG. 13C: recovered PLPs adhere to BSA and fibrinogen (FIB) with a characteristic tubulin ring, and spread extensively after activation with thrombin (green—beta tubulin, red—actin, blue—DNA). Scale bar=10 μm. Bioreactor conditions: 1.5 μL/min center channel and 0 μL/min outer combined flow rate.

FIGS. 14A-14C include representative results of expired blood platelets in the bioreactor compared to PLPs released from Mks, according to one embodiment of the present disclosure. FIG. 14A: profile of expired platelets per 5-min time interval within a bioreactor (counted three times). Error bars indicate ±SEM. FIG. 14B: images of Calcein-stained platelets flowing through bioreactor with either (i) 5 μL/min or (ii) 0 μL/min combined outer channel flow rate. FIG. 14C: images of Mks releasing PLPs from the slits with either (i) 5 μL/min and (ii) 0 μL/min combined outer channel flow rate. The center channel flow rate was maintained at 1.5 μL/min. Blue arrows indicate the flow direction. Yellow arrows indicate platelets or PLPs.

FIGS. 15A-15B include representative results of shear rate analysis with no outer channel flow, according to one embodiment of the present disclosure. FIG. 15A: shear rates through the entire region. FIG. 15B: details for individual slits for a flow rate of 1.5 μL/min in the center channel with 0 μL/min in the outer channels. White arrows indicate the flow direction. Estimated shear rates on proPLTs (dashed lines) are within 100 μm from the slits.

FIGS. 16A-16C include representative results of average CFD outputs across the slits along the x-axis, according to one embodiment of the present disclosure. Average pressure (FIG. 16A), velocity profile (FIG. 16B), and strain rate (FIG. 16C) across the slits for two combined outer channel flow rates. Blue=5 μL/min, Red=0 μL/min. The center channel flow rate was maintained at 1.5 μL/min. Dashed line on plots represent the 7-μm slit opening where velocity is the highest.

FIGS. 17A-17D include representative results of streamline observations under different outside channel flow rates, according to one embodiment of the present disclosure. CFD streamlines (FIG. 17A) and experimental streamlines (FIG. 17B) using 1-μm fluorescent beads and Calcein-stained cells for a flow rate of 1.5 μL/min in the center channel with 5 μL/min combined outer channel flow rate. CFD streamlines (FIG. 17C) and experimental streamlines (FIG. 17D) using 1-μm fluorescent beads and Calcein-stained cells for a flow rate of 1.5 μL/min in the center channel with 0 μL/min in the combined outer channel flow rate. White arrows indicate the flow direction. Scale bar=50 μm.

FIGS. 18A-18B include representative results of CFD streamlines overlaid on images from Mk experiments, according to one embodiment of the present disclosure. Center channel flow rate of 1.5 μL/min with 5 μL/min (FIG. 18A) and 0 μL/min (FIG. 18B) combined outer channel flow rate. Blue arrows indicate direction of flow. Black arrows indicate proPLTs. Yellow arrows indicate PLPs. Scale bar=50 μm.

FIGS. 19A-19B include representative results of differences in flow structures depicted with velocity vectors, according to one embodiment of the present disclosure. FIG. 19A: velocity vectors within slits for a flow rate of 1.5 μL/min in the center channel with a 5 μL/min combined outer channel flow rate. FIG. 19B: velocity vectors within slits for a flow rate of 1.5 μL/min in the center channel with a 0 μL/min combined outer channel flow rate. 20-μm spheres shown within slits simulate the effects of cells in the slits, while dashed lines represent direction of proPLTs/PLPs.

FIG. 20 includes a representative schematic diagram of the methodology underlying CFD stimulations, according to one embodiment of the present disclosure.

FIGS. 21A-21B include representative CFD analysis for a published bioreactor (i.e., Nakagawa et al.). FIG. 21A: mesh settings used to generate the displayed mesh. The geometry was slightly rotated to show the depth and elements across the slits. FIG. 21B: velocity profile of the reactor with insets of specific regions in the system. Inlet flow rate=16.7 μL/min for each channel. White arrows indicate direction of flow.

FIGS. 22A-22B include representative analysis of individual slit shear stress ranges for a published bioreactor (i.e., Nakagawa et al.). FIG. 22A: shear stress profile for the reactor. Inset shows the area of the slit that was designated high or low shear. FIG. 22B: range of shear stress values for selected slits. Inlet flow rate=16.7 μL/min for each channel. White arrows indicate direction of flow.

FIGS. 23A-23B include representative CFD analysis for a published bioreactor (i.e., Thon et al.). FIG. 23A: mesh settings used to generate the displayed mesh. The geometry has been slightly rotated to show the depth and elements across the slits. FIG. 23B: velocity profile of the bioreactor. Combined inlet flow rate=12.5 μL/hr. White arrows indicate direction of flow.

FIGS. 24A-24B include representative analysis of individual slit shear stress ranges for a published bioreactor (i.e., Thon et al.). FIG. 24A: Shear stress profile for the reactor. Numbers represent all the slits. Inset shows the area of the slit that was designated high or low shear. FIG. 24B: range of shear stress values for all the slits. Inlet flow rate=12.5 μL/hr. White arrows indicate direction of flow.

FIGS. 25A-25B include representative schematic diagrams of a bioreactor of the present disclosure. FIG. 25A: entire bioreactor. FIG. 25B: cropped region in the center to isolate the slit area.

FIGS. 26A-26B include representative results of CFD analysis of a bioreactor of the present disclosure. FIG. 26A: mesh settings used to generate the displayed mesh. The geometry has been slightly rotated to show the depth and elements across the slits. FIG. 26B: velocity profile of the reactor. Center channel inlet flow rate=1.5 μL/min and outer channels inlet flow rate=0.75 μL/min each. White arrows indicate direction of flow.

FIGS. 27A-27B include representative results of individual shear stress range of a bioreactor of the present disclosure. FIG. 27A: shear stress profile for the reactor. Numbers represent slits. Inset shows the area of the slit that was designated high or low shear. FIG. 27B: range of shear stress values for all the slits. Center channel inlet flow rate=1.5 μL/min and outer channels inlet flow rate=0.75 μL/min each. Log-scale. White arrows indicate direction of flow.

FIGS. 28A-28B include representative results of quantification of PLPs on proPLTs. FIG. 28A: images of Calcein-stained Mks extending proPLTs; black arrows indicate the PLPs that were counted on these proPLTs (0 μL/min center channel combined flow rate). FIG. 28B: images of Calcein-stained Mks extending proPLTs; white arrows indicate the PLPs that were counted on these proPLTs (5 μL/min center channel combined flow rate). Blue arrows indicate the flow direction.

FIGS. 29A-29B include representative results of PLP release kinetics in a bioreactor of the present disclosure. FIG. 29A: profile of PLPs released per 5-min time interval within bioreactors with center channel flow rate changes and observed cell blockages denoted by green arrow. FIG. 29B: profile of PLPs released per 5-min time interval within a bioreactor outer combined channel flow rate change and observed cell blockage denoted by green arrow. For FIG. 29A, combined outer channel flow rate=5 μL/min. For FIG. 29B, center channel flow rate=1.5 μL/min Color legend depicts the number of slits making PLPs per 5-min time interval.

FIGS. 30A-30B include representative results of pressure drop from CFD modeling in a bioreactor of the present disclosure. FIG. 30A: pressure drop values across the reactor from CFD simulations of completely blocking slits within a cell-free bioreactor with a flow rate of 1.5 μL/min in the center channel and a combined outer channels flow rate of 5 μL/min (line fitted to 1/# Slits Open). FIG. 30B: for a system with 2 open slits, pressure drop values from CFD simulations for different flow rates in the center channel with a constant combined flow rate of 5 μL/min in the outer channels (fitted to a straight line).

FIGS. 31A-31B include representative results of the number of PLP releases or active slits based on system changes in a bioreactor of the present disclosure. FIG. 31A: number of PLPs released in the 5-min time interval before or during a cell blockage event or a center channel flow rate increase. FIG. 31B: number of slits making PLPs in the 5-min time interval before and during a cell blockage event or a center channel flow rate increase. Combined outer channels flow rate maintained at 5 μL/min. Results represents 8 bioreactors runs across 3 different Mk cultures. Error bars±SEM.

FIGS. 32A-32B include representative results of bioreactor derived PLPs and pre-released particles analysis. FIG. 32A: Pre-released PLPs per 5-min interval (normal fit). FIG. 32B: Estimated percentage of PLPs that were generated from trapped Mks at the slits. Results represent four bioreactor runs across two different Mk cultures. Center channel flow rate=1.5 μL/min and combined outer channel flow rate=0 μL/min.

FIGS. 33A-33D include representative results of CFD analysis across slits along the x-axis and center height (z=20 μm) in a bioreactor of the present disclosure. FIG. 33A: slit location for analysis. FIG. 33B: pressure along individual slits for two different outer channel combined flow rates (5 μL/min and 0 μL/min). FIG. 33C: individual slit velocities along the x-axis for a combined outer channel flow rate of 5 μL/min. FIG. 33D: individual slit velocities along the x-axis for a combined outer channel flow rate of 0 μL/min. For all outputs, the center channel flow rate was maintained at 1.5 μL/min. White arrows indicate the flow direction. Slit numbers shown in FIG. 33A are indicated by number and line colors, as indicated in the legend below the figure.

FIG. 34 includes a representative diagram of an overall perspective view of the USRB-5 μm system, according to one embodiment of the present disclosure.

FIG. 35 includes a representative diagram of the USRB-5 μm system that includes the geometric relationships of a central flow channel and two branching arms, according to one embodiment of the present disclosure.

FIG. 36 includes a representative diagram of the USRB-5 μm system that includes slit channels with 3 rows of 5.5-μm slits, according to one embodiment of the present disclosure.

FIG. 37 includes a representative diagram of the USRB-5 μm system that is scaled by adding more rows (increasing the width of the reactor) and/or by adding more branches to the arms (increasing the length of the reactor), according to one embodiment of the present disclosure.

FIG. 38 includes representative image analysis of CFD modeling of the USRB-5 μm system, according to one embodiment of the present disclosure.

FIG. 39 includes representative diagrams modeling the production of platelets and proplatelets using the USRB-5 μm system, according to one embodiment of the present disclosure.

FIG. 40 includes a representative image obtained using fluorescent beads and Calcein-stained cells to allow for the visualization of system dynamics of the USRB-5 μm system, according to one embodiment of the present disclosure.

FIG. 41 includes representative data obtained using mobilized peripheral blood (mPB) Mks to characterize the USRB-5 μm system, according to one embodiment of the present disclosure.

FIG. 42 includes representative data of recirculation experiments obtained using cord blood cells to characterize the USRB-5 μm system, according to one embodiment of the present disclosure.

FIG. 43 includes representative schematic diagrams of a scaled-up process for generating PLPs, according to one embodiment of the present disclosure.

FIG. 44 includes representative images of a USRB-5 μm system as compared to a USRB-7 μm, according to one embodiment of the present disclosure.

FIG. 45 includes a representative image of a USRB-7 μm with CB-Mks. Center channel flow: 1.5 μL/min Combined outside flow: 0 μL/min. Green arrow indicate direction of flow. CB-Mks have been calcein stained.

FIGS. 46A-46B include representative results of USRB-5 μm computational fluid dynamics modeling. FIG. 46A: a narrow-slit system was modeled using CFD to understand and confirm the uniform shear environment. FIG. 46B: the flow profile was also analyzed using predicted streamlines. Center channel flow: 1.5 μL/min Combined outside flow: 0 μL/min. White arrow indicate direction of flow. The CFD analysis was carried out in the same manner as the USRB-7 μm in Martinez et al. (2017).

FIGS. 47A-47B include representative images of a USRB-5 μm loaded with CB-Mks. FIG. 47A: representative image of bioreactor runs for three different culture conditions (C1, C2 and C3) showing calcein-stained selected Mks generating proPLTs and PLPs (blue arrows). FIG. 47B: video analysis of the bioreactor runs allowed estimation of the cumulative rate of PLP-release for each bioreactor shown in FIG. 47A and to distinguish between better performing conditions. Center channel flow: 1.5 μL/min Combined outside flow: 0 μL/min. White arrow indicate direction of flow.

FIGS. 48A-48B include representative images of a fibrinogen coated USRB-7 μm. FIG. 48A: image of a fluorophore-tagged fibrinogen coated bioreactor. FIG. 48B: CB-Mks (top) and mPB-Mks (bottom) introduced into a fibrinogen-coated bioreactor. FIG. 48C: comparison of mPB-Mks in uncoated, fibrinogen, and fibronectin coated reactors showing that there is greater retention and capture of cells behind the slits. Blue and yellow arrows indicate proplatelets and PLPs. Mks are stained with calcein. Center channel flow: 1.5 μL/min Combined outside flow: 0 μL/min. White arrow indicate direction of flow.

FIGS. 49A-49B include representative results of protein coated bioreactors and PLP kinetics for mPB-Mks, according to one embodiment of the present disclosure. FIG. 49A: plot shows the cumulative PLP release over the perfusion runs through video analysis as described in Martinez et al. (2017). Yellow line=Day 10. Blue line=Day 11. Red Line=Day 12. Control=yellow triangles. Fibrinogen (Fib)=blue dots. Fibronectin (Fn)=green squares. FIG. 49B: summary table of the kinetics for each day and condition and estimates on the number calcein particles observed in the video analysis. Day 10 and 11, control reactors have the lowest PLP production compared to protein coated reactors. Fibrinogen on Day 11 has the highest production across all the days and conditions. On Day 12, the uncoated control reactor reaches high PLP levels, similar to levels observed of Fib and Fn. Protein coatings appear to help early PLP release before Mks are more mature on Day 12.

### DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As used herein and in the appended claims, the singular forms “a”, “an” and “the” include plural reference unless the context clearly dictates otherwise. Thus, for example, reference to “a device” is a reference to one or more devices and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### DETAILED DESCRIPTION

The present disclosure relates to the production of platelets from megakaryocytes (Mks). In particular, the present disclosure provides systems and methods for the in vitro production of platelets from Mks using a microfluidic bioreactor having a center flow channel and uniform high-shear micrometer slits. Use of this microfluidic bioreactor enables the continuous production of millions of platelets and facilitates real-time and long-term visualization of proplatelet and platelet generation.

In accordance with the various embodiments of the present disclosure, mature megakaryocytes (Mks) can be continuously introduced into a microfluidic bioreactor using a single flow that distributes itself through smaller branching arms where slits capture the megakaryocytes. The microfluidic bioreactor causes the megakaryocytes to experience shear forces to produce proplatelets and platelets. The microfluidic bioreactor is designed to mimic the lung capillary bed in which branching of larger vessels into smaller channels lead Mks into high shear regions at the slits; hence, it is referred to as “Lung-USRB” (lung uniform-shear rate bioreactor).

Embodiments of the present disclosure involve the application of microfluidic fabrication technology to develop the bioreactor system. In accordance with these embodiments, platelet-like-particles with functional activity have been generated from megakaryocytes introduced into the system. Currently, processed Mk numbers range from about 200K up to about 800K, and are introduced as a single bolus injection. Using an Mk reservoir that continuously supplies a source of Mks can generate millions of Mks over a period of hours.

Embodiments of the present disclosure provide improvements over typical systems and methods for the in vitro production of platelets and proplatelets. In accordance with these embodiments, the present disclosure includes slit bioreactor systems (termed “USRB” for uniform-shear rate bioreactor) that are improvements over previous published slit bioreactors by allowing real-time and long-term visualization of the proPLT formation process within the entire reactor. Embodiments of the USRBs of the present disclosure were filed as U.S. Provisional Application Ser. No. 62/522,491 and U.S. Provisional Patent Application Ser. No. 62/642,955, which are both herein incorporated by reference in their entireties and for all purposes.

Additionally, because the shear rate within currently available slit bioreactor systems was not fully characterized, computational fluid dynamics (CFD) modeling was used to develop the USRBs of the present disclosure and to increase understanding of the platelet formation process. The environment promoted the rapid release of individual PLPs from Mks in the USRBs of the present disclosure, which has been reported in vivo but not in bioreactors in vitro. Initially, the USRBs were designed to mimic the endothelial gaps and fenestrations in the bone marrow, where a primary flow pushes the Mks into slits and a secondary flow exerted shear forces on extending proplatelets, similar to the in vivo process. However, further investigations determined that a single primary flow on the Mks was six-times more efficient at generating proplatelets and platelets and most previously published systems utilized a two-flow set-up. Recent studies have shown that Mks can enter the blood flow and get trapped in the capillary bed of the lung where high shear forces (>2000 s⁻¹) elongate and fragment Mks into platelets within minutes.

Therefore, based on observations of platelet generation in the lung and on data generated using a single-flow system, the USRBs of the present disclosure were modified and scaled up into the “Lung-USRB” system (USRB-5 μm). This novel bioreactor systems of the present disclosure include key features, such as the post geometries, but additionally uses slits with various sizes (e.g., 5-7 μm) to improve capture and to allow flexibility of using Mks derived from different cell sources. For example, as disclosed herein, use of only a center flow channel without the outside flow channels of a USRB-7 μm system, in combination with 5-μm slits of the USRB-5-μm system, led to a surprising and unexpected 6-fold increase in proplatelet production. The USRB-5-μm system with 5-μm slits maintained uniform shear rates with at least a 90-fold increase in capture area, as compared the USRB-7 μm system. Both the USRB-5-μm system and the USRB-7 μm system provide significant advantages over other in vitro systems and methods for platelet generation; however, determining which USRB system to use will depend on various factors, such as particular clinical and/or laboratory needs, as would be recognized by one of ordinary skill in the art based on the present disclosure.

Embodiments of the present disclosure include the use of CFD to guide platelet bioreactor design, analyze forces on Mks, and examine bioreactor performance. In some embodiments, CFD was used to evaluate published slit bioreactors and develop a USRB system design with improved flow and shear uniformity. The 4-μm slit bioreactor introduced by Nakagawa et al. had no specified shear rates. CFD modeling disclosed herein determined rates on proPLTs (along the length of the bioreactor) were within the sinusoids range (200 s⁻¹) and above physiological rates (6,000 s⁻¹), depending on the location. However, the shear rate within the 4-μm slits where Mks would be trapped had a much higher range of 400-30,000 s⁻¹. The substantial nonuniformity of shear rates along the bioreactor length make it difficult to study proPLT/PLP formation real-time since Mks at different regions of the bioreactor would experience substantially different microenvironments.

The 2-μm slit bioreactor developed by Thon et al. had a narrower range of shear rates than Nakagawa et al. Modeling disclosed herein used CFD to analyze this system and determined a shear rate of approximately 500 s⁻¹. CFD outputs showed that, along the length of the bioreactor, proPLTs would experience shear rates from 250 to 500 s⁻¹, and that the slit shear rates ranged from 5,000 to 7,500 s⁻¹. Additionally, Thon et al. provided real-time visualization of the proPLT formation process. However, the shear rates within the slits showed a steady increase along the bioreactor so that Mks trapped at various slits would be exposed to different shear rates.

CFD analysis of the Nakagawa et al. and Thon et al. systems suggests the shear rate across slits increased along the bioreactor from the inlet to the outlet. This can be attributed to the design of the systems in which two parallel-like flows are separated by slits and where the top flow (pushing on the Mks) is redirected into the lower channel at the end of the bioreactor length (Table 1). To avoid generating this increase of shear rates across the slits, USRBs of the present disclosure were instead designed with the two outer flows converged at a 90° C. V-shaped region. This arrangement allowed the center channel flow to push whole-Mk bodies into the 5-7-μm slits with similar maximum shear rates of 5,000 s⁻¹. The outer flow converges at the slits and exerts nearly uniform shear rates (250-350 s⁻¹) on extending proPLTs. Mks exiting the bone marrow sinusoids can be trapped in the vascular bed of the lung where high shear forces are exerted on the whole cell body and on proPLTs, thus, high shear forces were utilized on Mk bodies and physiological shear on the proPLTs. CFD analysis was extended to include cell blockages within the slits and confirmed the flow patterns experimentally to understand behavior of an occupied bioreactor and to estimate anticipated shear rates on proPLTs (100-900 s⁻¹).

Compared to Thon et al., the USRBs of the present disclosure have a similar capture area of 20 slits vs. 15 slits, but a higher slit occupancy (90% vs 66%). More importantly, the slits can be observed during an experimental run and it was noted that on average 40-60% of occupied slits were actively making proPLTs/PLPs. In contrast, the length of the Thon et al. and Nakagawa et al. bioreactors makes it difficult to analyze proPLT/PLP formation from all the slits at the same time. In addition to supporting proPLT production, the USRBs of the present disclosure also promoted rapid release of many individual PLPs, which has not been reported for other published bioreactor systems. This observation is physiologically relevant since Mks have also been observed to make platelets in vivo (in mouse) via a rapid fragmentation process that releases platelets without the proPLT formation step. It was observed that the rate of PLP production appears to be faster when PLPs are rapidly released (Video 1 vs. Video 2). It may be that rapid PLP generation within the USRBs with 5-7 μm slits is largely influenced by the unique slit geometry in which cells are pushed through a hyperbolic-like-converging region. As the area is reduced in this region, whole Mks bodies are squeezed and elongated through the 5-7-μm gap where the shear rate and strain rate are the highest.

Results of the present disclosure show that flow microenvironment can greatly affect the behavior of Mks in real-time. Within the system, it was observed that Mk capture at an open slit increased the release of PLPs across the other Mk-blocked slits. Slit-blockage events could be influenced by the size of the Mks being trapped and are not easily controlled, thus, these observations highlight the importance of understanding how the inherent dynamics of a bioreactor can impact the Mk response. A step-change in the center channel flow rate (while keeping the outside channel flow constant) transiently increased the rate of PLP releases, similarly to the slit-blockage events. The increase in immediate Mk productivity could be attributed to an increase in pressure drop across the slits, as presented by CFD analysis. While a temporary three-fold increase in productivity was observed after cell-blockages or flow rate changes in the USRBs of the present disclosure, it was recognized that the rates are not sustained for the remainder of an experimental run largely due to the dynamic behavior of cell capture and slit openings. Nagakawa et al. did not study the effect of changing the flow conditions within their system. Thon et al. found that the average proPLT extension rate did not change at different flow rates (same flow for both channels, 12.5-100 μL/h), but did not extend their CFD analysis to other flow regimes. Within the USRBs with 5-7 μm slits, it was observed that step-changes to the center channel flow rate or a cell-blockage event led to approximately 30% and approximately 50% increases in active slits, respectively.

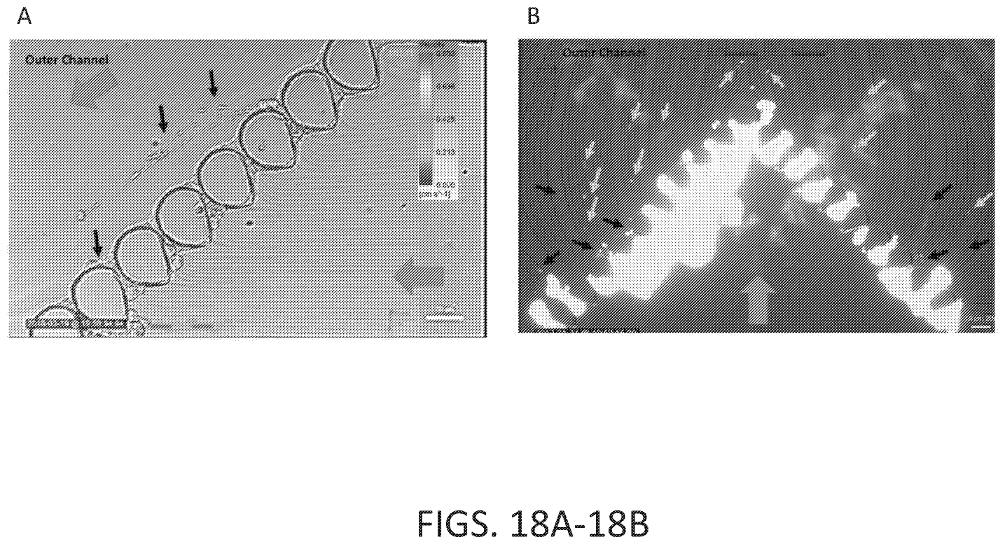

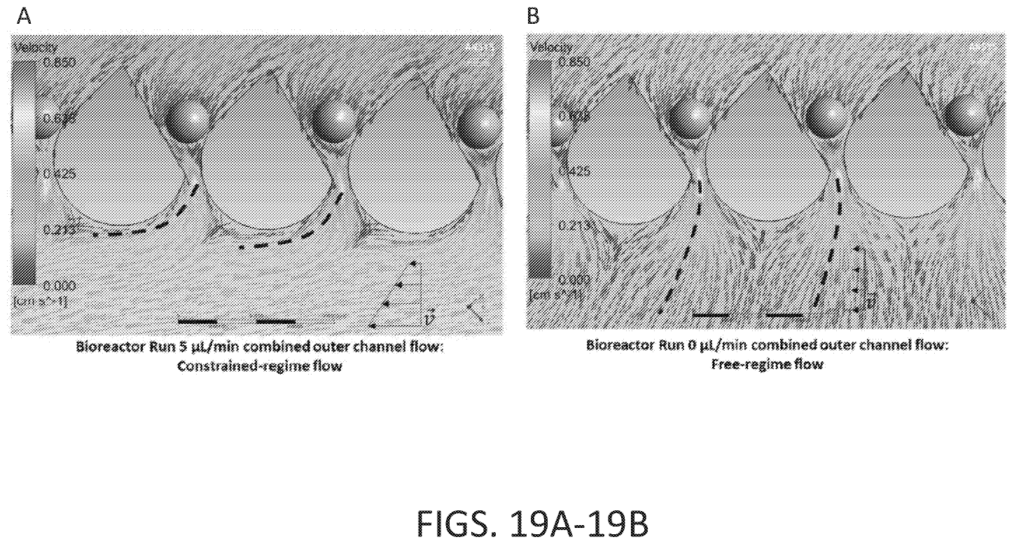

It was expected that the presence of an outside channel flow would aid in shearing off proPLTs (increasing the PLP release rate), mimicking physiological blood flow in vivo. Thus, it was unexpected that turning off the outside channel flow rate increased the average number of PLPs released by almost 6-fold. Using CFD simulations, the predicted environment was investigated to try and understand the variables responsible for these unforeseen results. Average velocity profiles, strain rates, and wall shear rates through the slits remained overall unchanged with or without flow in the outer channel. CFD predictions showed a slight increase in back-pressure by the outer flow on the center channel, which could inhibit Mks from releasing more PLPs. However, a significant change in the flow structure was observed, as confirmed by correlations between the CFD streamlines and experimental streamlines. The CFD simulations showed that the slits appear to operate independently from each other with no outside flow (FIG. 19), and within these experimental runs, it was observed that Mk behavior that supported the simulations in which proPLTs and PLPs were not contracted toward the center of the bioreactor (FIG. 18B vs. FIG. 18A). Thus, it is likely that the flow structure had the most significant impact on Mk behavior.

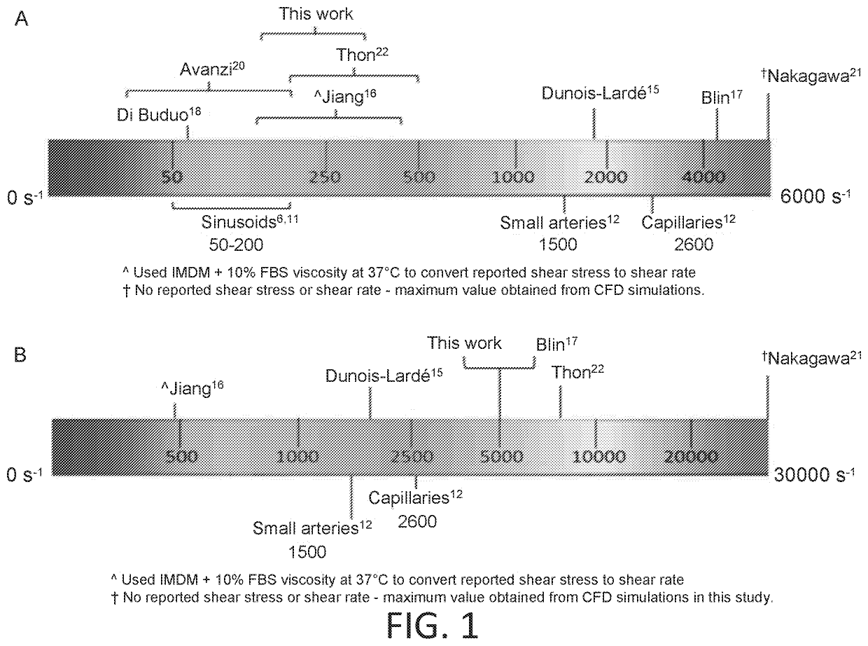

Analysis of the bioreactor effluent showed CD41⁺CD42b⁺ PLP populations that exhibited activation following thrombin addition. Video analysis indicated that approximately 76% of Calcein-stained particles were generated at the slits in reactors with no outside flow. ProPLTs are subjected to a shear environment typical of the bone marrow sinusoids in many of the current published bioreactor systems (FIG. 1A). Additionally, Mk bodies can be directly exposed to high shear environments that approach and exceed estimated values within the lung (FIG. 1B). Single flow environments that transport Mks into regions with high shear forces (operating similar to the lung capillary bed) as well as extensional forces are sufficient for PLP generation, as demonstrated by the results of the present disclosure, challenge the need of using two flows to mimic the bone marrow niche.

Section headings as used in this section and the entire disclosure herein are merely for organizational purposes and are not intended to be limiting.

### 1. Bioreactor Design

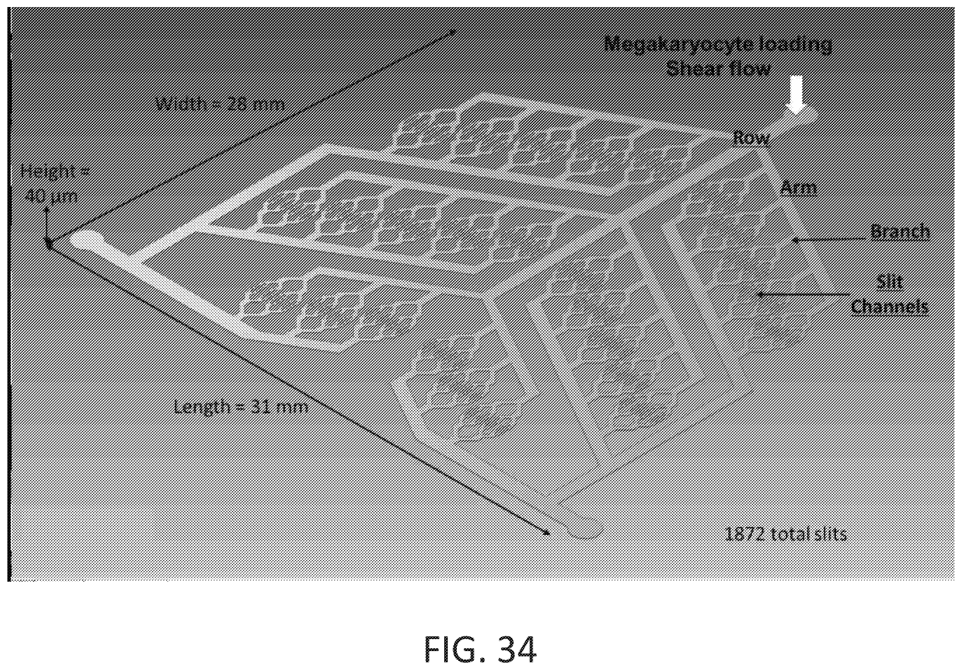

Embodiments of the USRB systems of the present disclosure include a main or central flow channel entering the USRB that splits into rows that contain left and right arms (branching flow channel). These branching arm channels can have parallel branches that lead to platelet production chambers (proPLTs/PLPs), which include smaller slit channels (FIG. 34). The slit channels in the platelet production chambers include a plurality of slits configured to capture a megakaryocyte and allow for the application of uniform shear stress to the megakaryocyte to facilitate the production of proPLTs/PLPs.

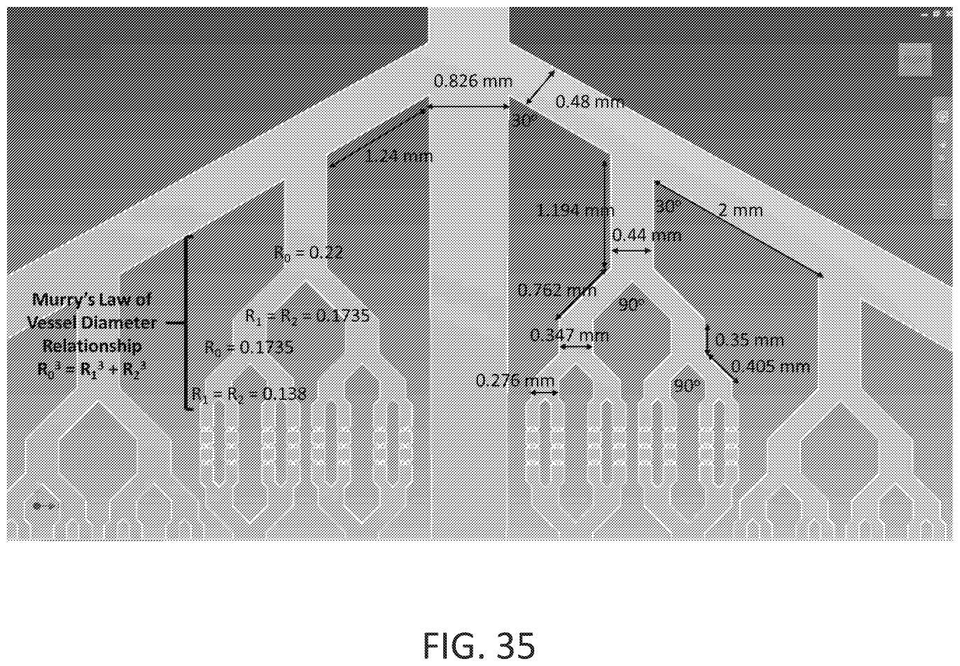

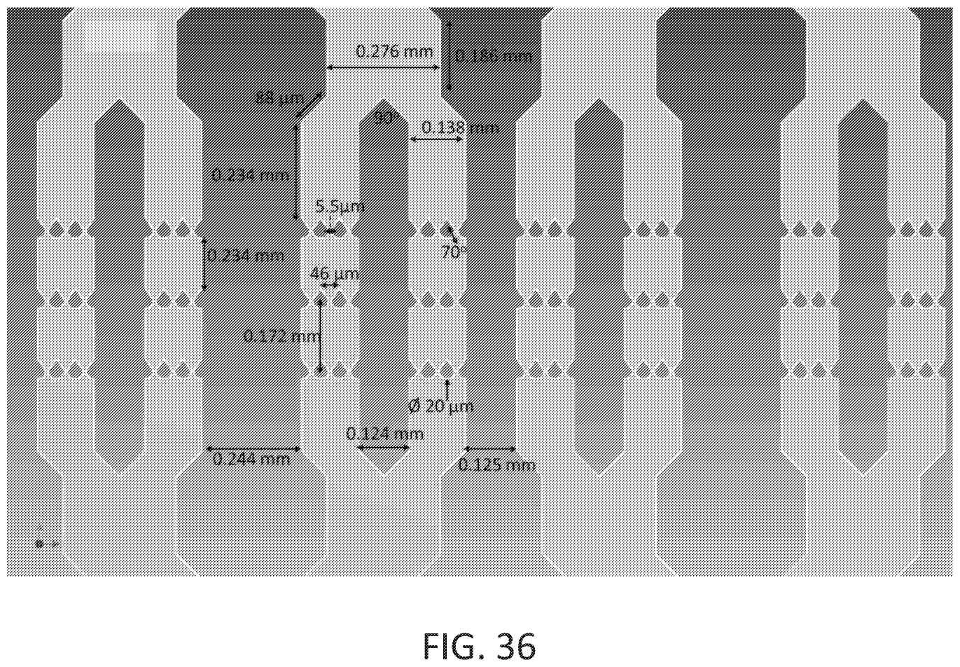

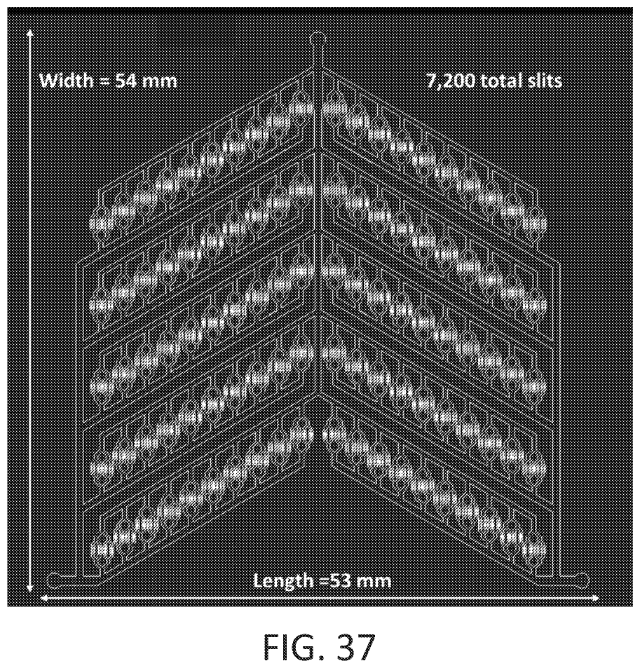

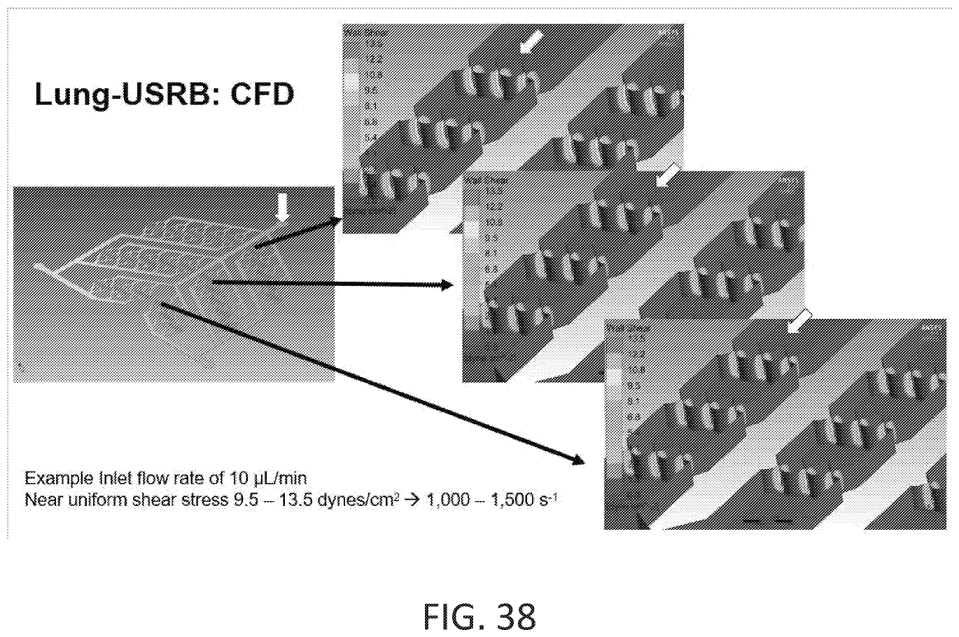

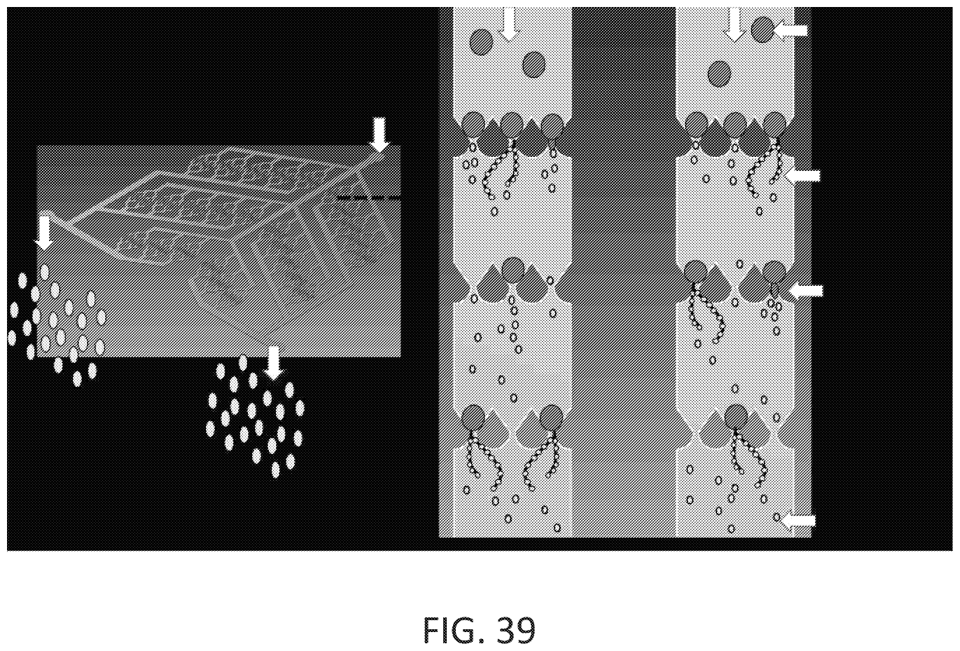

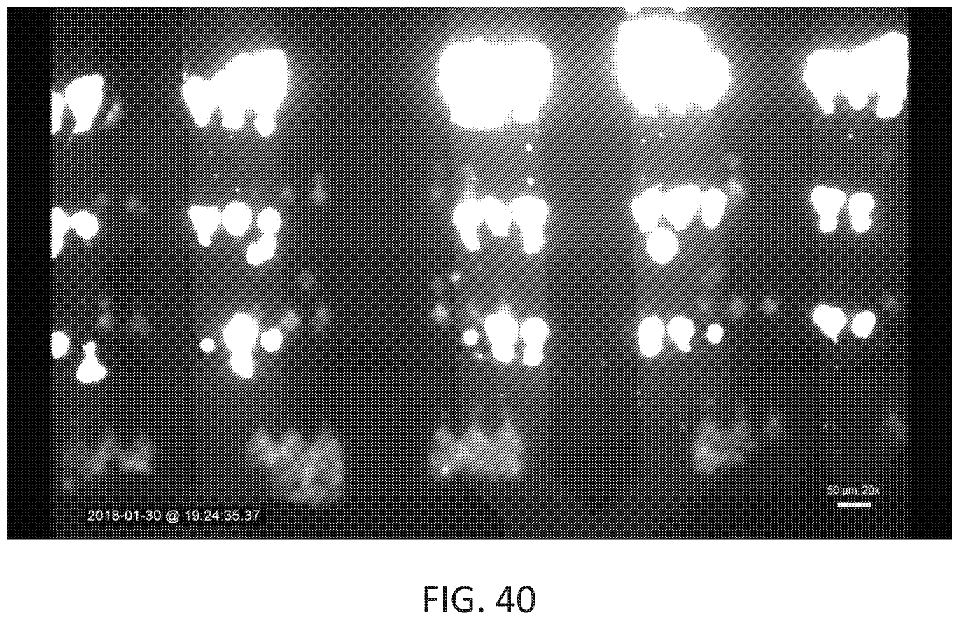

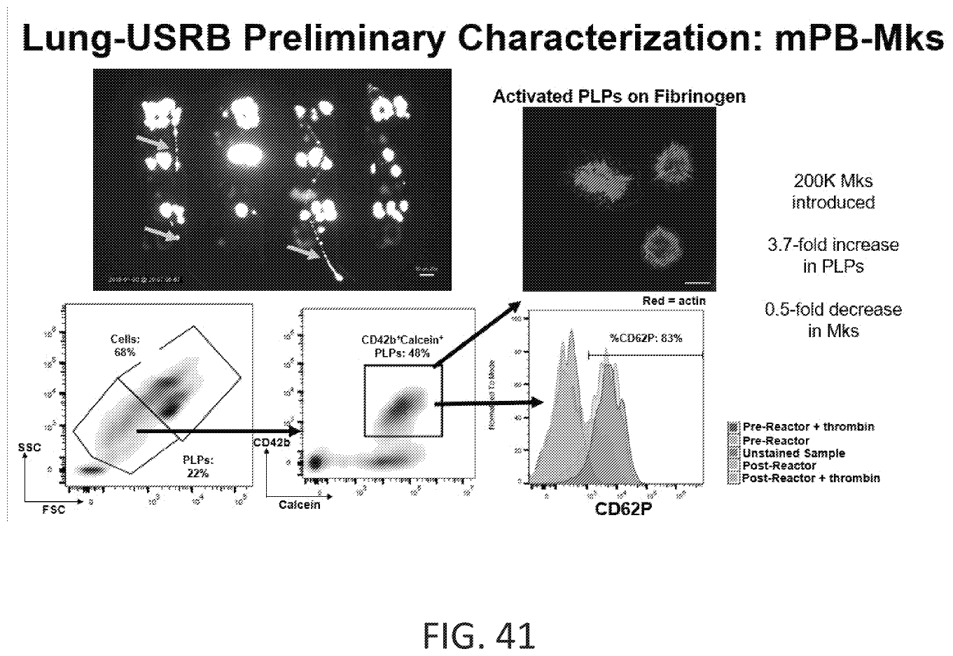

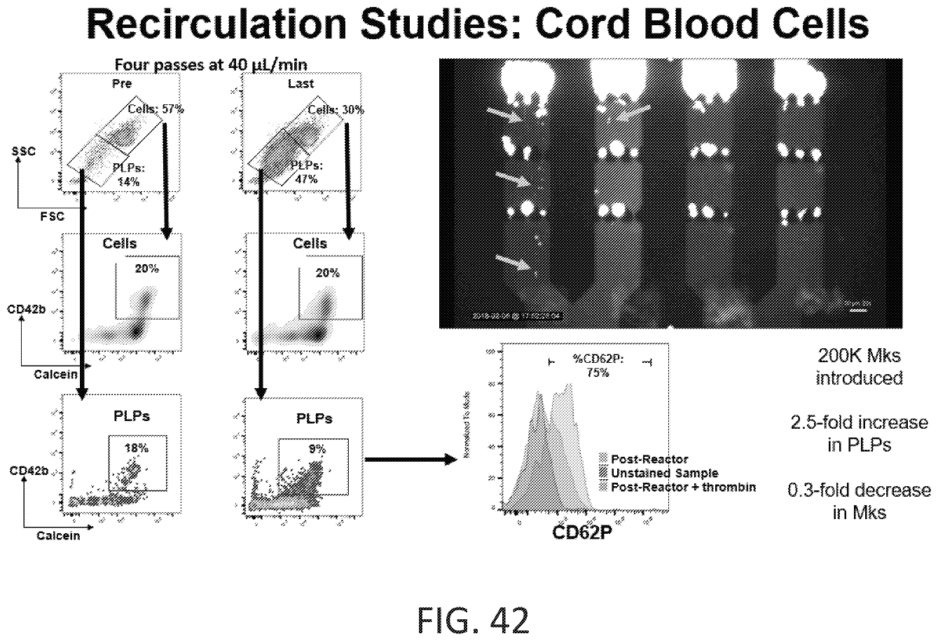

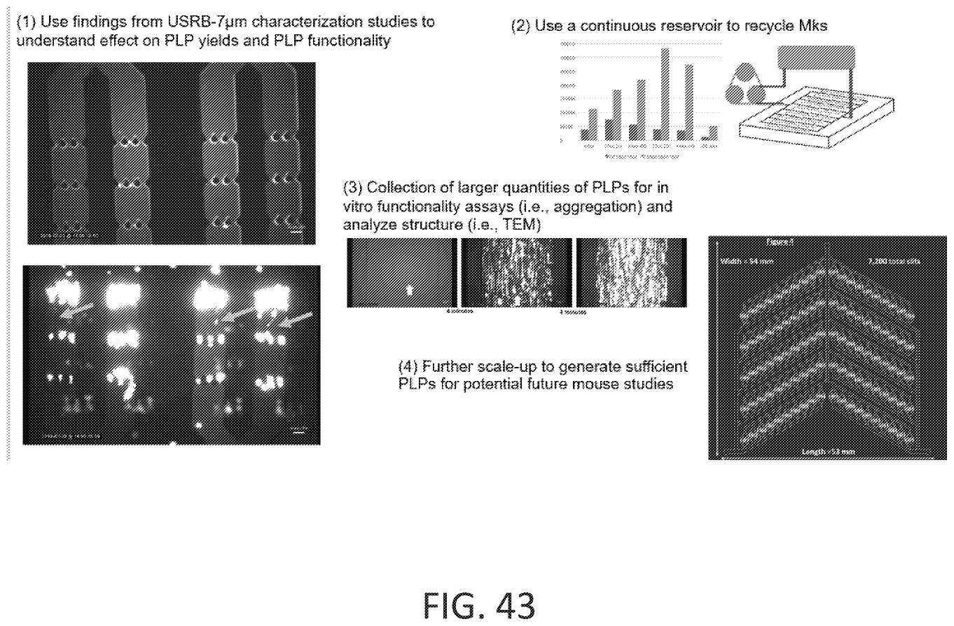

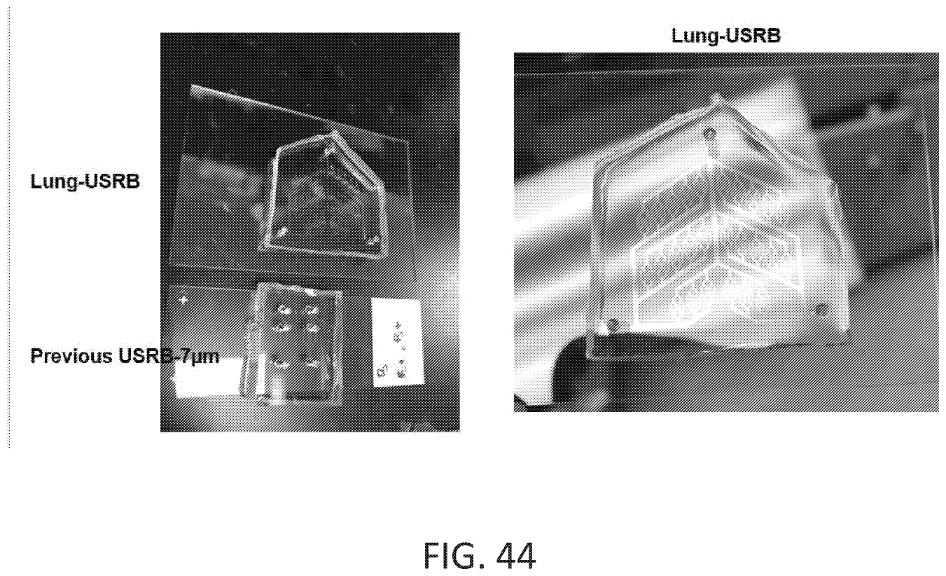

In some embodiments, the layout of the USRB systems of the present disclosure use Murray's law that describes vessel diameters and branching, where the radius of the parent branch (Rₒ) is related to the sum of the radius of the daughter branches (R₁, R₂): Ro³=R₁³+R₂³. Thus, based on this relationship, a geometry of the USRB systems disclosed was defined as shown in FIG. 35. The branching angles were set at 30°. Within the slit channels are 3 rows of 5.5-μm slits (FIG. 36). The layout of the system allows it to be scaled further by adding more rows (increasing the width of the reactor) and/or by adding more branches to the arms (increasing the length of the reactor) (FIG. 36). The slit dimension was chosen to retain mature Mks, which are typically greater than 20 μm. The height of the reactor was chosen to be 40 μm, which is similar to blood sinusoid dimensions.

Embodiments of the present disclosure include microfluidic proplatelet (proPLT) and platelet-like particle (PLP) production chamber devices and systems. In accordance with these embodiments, the device includes a plurality of slit channels comprising one or more proPLT/PLP production slits. In some embodiments, the slits can range in width from about 3.0 μm to about 10 μm, from about 4.0 μm to about 10 μm, from about 5.0 μm to about 10 μm, from about 6.0 μm to about 10 μm, and from about 8.0 μm to about 10 μm. In some embodiments, the slits can range from about 3.0 μm to about 9.0 μm, from about 4.0 μm to about 8.0 μm, and from about 5.0 μm to about 7.0 μm. In some embodiments, the slits can range from about 5.0 μm to about 6.0 μm, including about 5.5-μm.

In some embodiments, the proPLT/PLP production devices and systems of the present disclosure is configured to expose a megakaryocyte to a uniform shear profile to facilitate the production of proPLTs/PLPs. As described herein, uniform shear profiles are based on a number of variables associated with microfluidics in general, and based on certain aspects of the proPLT/PLP production devices of the present disclosure. For example, a uniform shear profile can include a maximum shear rate at a proPLT/PLP production slit. In some embodiments, a uniform shear profile can include a maximum shear rate at one or more proPLT/PLP production slits ranging from about 5000 s⁻¹ to about 10,000 s⁻¹, from about 5,000 s⁻¹ to about 9,000 s⁻¹, from about 5,000 s⁻¹ to about 8,000 s⁻¹, from about 5,000 s⁻¹ to about 7,000 s⁻¹, from about 5,000 s⁻¹ to about 6,000 s⁻¹, from about 5,000 s⁻¹ to about 7,500 s⁻¹, from about 7,000 s⁻¹ to about 10,000 s⁻¹, from about 8,000 s⁻¹ to about 10,000 s⁻¹, or from about 9,000 s⁻¹ to about 10,000 s⁻¹.

In some embodiments, the uniform shear profile includes a maximum shear rate at one or more proPLT/PLP production slits of about 5000 s⁻¹ when the slits are about 7 μm wide. In other embodiments, the uniform shear profile includes a maximum shear rate at one or more proPLT/PLP production slits of about 8,200 s⁻¹ when the slits are about 5 μm wide.

In some embodiments, a uniform shear profile can include a shear rate on proPLTs/PLPs being produced that ranges from about 50 s⁻¹ to about 500 s⁻¹, from about 100 s⁻¹ to about 500 s⁻¹, from about 150 s⁻¹ to about 500 s⁻¹, from about 200 s⁻¹ to about 500 s⁻¹, from about 250 s⁻¹ to about 500 s⁻¹, from about 300 s⁻¹ to about 500 s⁻¹, from about 350 s⁻¹ to about 500 s⁻¹, from about 400 s⁻¹ to about 500 s⁻¹, from about 450 s⁻¹ to about 500 s⁻¹, from about 50 s⁻¹ to about 300 s⁻¹, from about 50 s⁻¹ to about 200 s⁻¹, from about 50 s⁻¹ to about 100 s⁻¹, from about 100 s⁻¹ to about 400 s⁻¹, from about 100 s⁻¹ to about 300 s¹, from about 200 s⁻¹ to about 500 s⁻¹, from about 200 s⁻¹ to about 400 s⁻¹, from about 200 s⁻¹ to about 300 s⁻¹, from about 250 s⁻¹ to about 500 s⁻¹, from about 250 s⁻¹ to about 450 s⁻¹, from about 250 s⁻¹ to about 400 s⁻¹, or from about 250 s⁻¹ to about 350 s⁻¹.

In some embodiments, the uniform shear profile includes a shear rate on the proPLTs/PLPs ranging from about 250 s⁻¹ to about 350 s⁻¹ when the one or more proPLT/PLP production slits are about 7 μm wide. In other embodiments, the uniform shear profile includes a shear rate on the proPLTs/PLPs ranging from about 50 s⁻¹ to about 100 s⁻¹ when the one or more proPLT/PLP production slits are about 5 μm wide.

In some embodiments, the proPLT/PLP production devices and systems of the present disclosure include a plurality of slit channels that are distal to and in fluid communication with a central flow channel. In some embodiments, fluid flows in a single direction from the central flow channel to the plurality of slit channels, rather than in a dual flow manner. In accordance with these embodiments, a single direction fluid flow can produce at least a 5-fold increase in proPLT/PLP production, and in some cases, at least a 6-fold increase in proPLT/PLP production.

In some embodiments, the proPLT/PLP production devices and systems of the present disclosure include a proPLT/PLP collection reservoir into which proPLTs/PLPs can be collected after formation. The proPLT/PLP collection reservoir is generally the most distal component of the present devices and systems, and in some cases, more than proPLT/PLP collection reservoir can be used. In some embodiments, the proPLT/PLP production devices and systems of the present disclosure include a megakaryocyte loading reservoir coupled to a central flow channel, and in some cases, megakaryocyte loading reservoir is the most proximal component of the present devices and systems. As described herein, a plurality of branching flow channels are located between the megakaryocyte loading reservoir and the proPLT/PLP collection reservoir. In accordance with these embodiment, the present devices and systems can also include a fluid source coupled to the central flow channel and configured to supply fluid pressure to the system to facilitate proPLT/PLP production.

Embodiments of the present disclosure also include methods for producing proPLTs/PLPs using the devices and systems described above, as would be recognized by one of ordinary skill in the art.

### 2. Bioreactor Fabrication

Embodiments of the present disclosure also include methods of manufacturing a USRB device and/or system, as well as a proPLT/PLP production chamber. Material and methods for manufacturing or fabricating the proPLT/PLP production devices and systems of the present disclosure are generally known to one of ordinary skill in the art. For example, a 2D design of the bioreactor was created in AutoCAD 2014 and then printed onto a chrome mask. A silicon wafer was spin-coated with SU8-2035 photoresist at 4000 RPM for 30 s to achieve a photoresist height of 40 μm. The wafer was soft-baked at 65° C. for 3 min and then hard-baked at 95° C. for 6 min. Afterwards, the wafer was exposed to UV light, using a Karl Suss MA6 Mask Aligner, for 17 s. The exposed resist was placed at 95° C. for 6 min (post-exposure bake).

The resist was developed using SU8 developer solution for 2 min and dried with a nitrogen gun. The dry wafer was silanized overnight in a vacuum chamber. Next, a 1:10 curing agent to polydimethyl siloxane (PDMS) solution was poured over the wafer to cast a mold that was placed in an oven at 65° C. overnight. The PDMS mold was then cut, holes for inlets and outlets punched with a 2-mm punch, and the PDMS plasma-bonded to an ethanol-cleaned premium plain glass slide.

As one of ordinary skill in the art would recognize based on the present disclosure, other materials and methods may be used to manufacture or fabricate one or more components of the proPLT/PLP production devices and systems of the present disclosure.

### 3. Materials and Methods

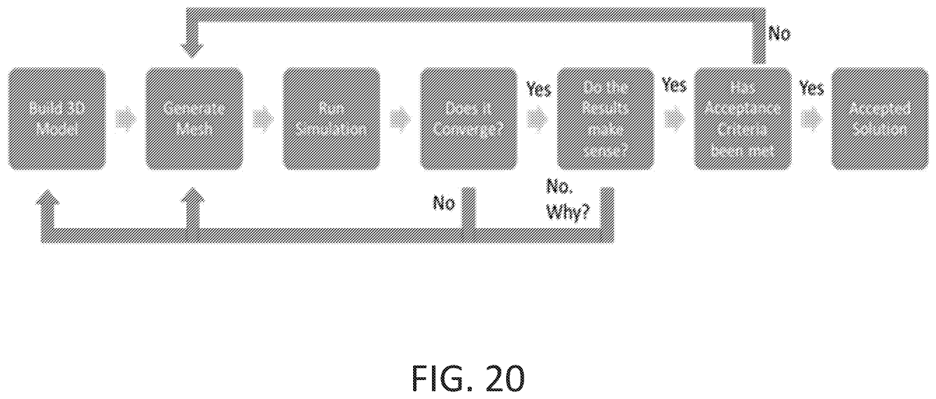

CFD Modeling. Flow simulations were conducted using ANSYS v16.1 (ANSYS, Inc., Canonsburg, Pa.). 3D models of the slit bioreactors were created using Autodesk Inventor Professional software 2015 (San Rafael, Calif.) and the files imported into ANSYS Design Modeler. A computational grid (mesh) was individually optimized for each system (FIG. 20). Boundary conditions were no-slip at the walls, constant inlet velocity, and default gauge pressure of 0 Pa at the outlet.

A summary of platelet slit bioreactors published to date is provided in Table 1. A description of the system and operation, as well as key dimensions, is given for each bioreactor. CFD modeling is presented below for the slit-type bioreactors.

Methodology. The slit bioreactor simulations were carried out using ANSYS version 16.1 (Canonsburg, Pa.) that includes the computational fluid dynamics solver FLUENT. The 3D models were created in Autodesk Inventor Professional software 2015 (San Rafael, Calif.). The files were converted to Parasolid binary text in Inventor and then imported into ANSYS Design Modeler. A mesh was then created for each system, in which the geometry is discretized into small volumes (elements) where the CFD calculates an approximate solution to the discretized form of the governing equations. The mesh for each individual system was optimized to yield a converging solution that is mesh-independent. The acceptance criteria used was a change in the predicted CFD velocity of no more than 5% from the previous converged solution. Boundary conditions were no-slip at the walls, constant inlet velocity, and default gauge pressure of 0 Pa at the outlet. The velocity input into each system was determined from the volumetric flow rate and the dimensions specified by the authors. FLUENT was used to solve the steady-state form of the Navier-Stokes Equation (Eq. 1) for an incompressible Newtonian fluid subjected to the specified flow conditions. The convergence tolerance for all simulations, which is the normalized residual for each degree of freedom, was set to 10⁻³. The overall methodology is shown in FIG. 20.

$$ρ ( {v}^{} \cdot {∇}^{} {v}^{} ) = - {∇}^{} P + μ {∇}^{} {}^{2} {v}^{} \\ ρ = density [ \frac{kg}{m 3} ] , v = velocity [ \frac{m}{s} ] , \\ μ = viscosity [ P a \cdot s , ] \\ P = pressure [ P a ] \qquad Eq . S1$$

Overall, the flow rates and designs lead to laminar flow conditions inside the bioreactors, such that viscous forces dominate inertial forces. Thus, the viscosity of the media used for perfusion through the systems and the operating temperature have a substantial impact on the expected shear forces within the bioreactors. Table 2 shows viscosity measurements for different media at 37° C. The measured fluid viscosity was used in the simulations.

TABLE 2
Viscosity measurements conducted with a Cannon-Fenske viscometer.

| | IMDM | IMDM + 20% BIT | IMDM + 10% FBS |
|---|---|---|---|
| Vis- | 0.0082 +/− 0.00008 | 0.0088 +/− 0.00005 | 0.0083 +/− 0.00005 |
| cosity | | | |
| dynes- | | | |
| s/cm² | | | |
| System | Nakagawa et al. | Present Disclosure | — |
| Used | | | |

Thon et al. provided media measurement of 0.012 dynes-s/cm².

All simulations for the systems were run with the following computer and software settings. Computer Specs: Dell Precision T1700, Intel® Core™ i7-4790 CPU @ 3.60 GHz, 32 GB RAM, 64-bit, Windows 10 Pro.

FLUENT Settings:
Solver: 3D, double-precision, pressure-based, parallel (8 processors)
Time: steady-state
Pressure-Velocity Coupling Scheme: Simple
Discretization: Second-order upwinding

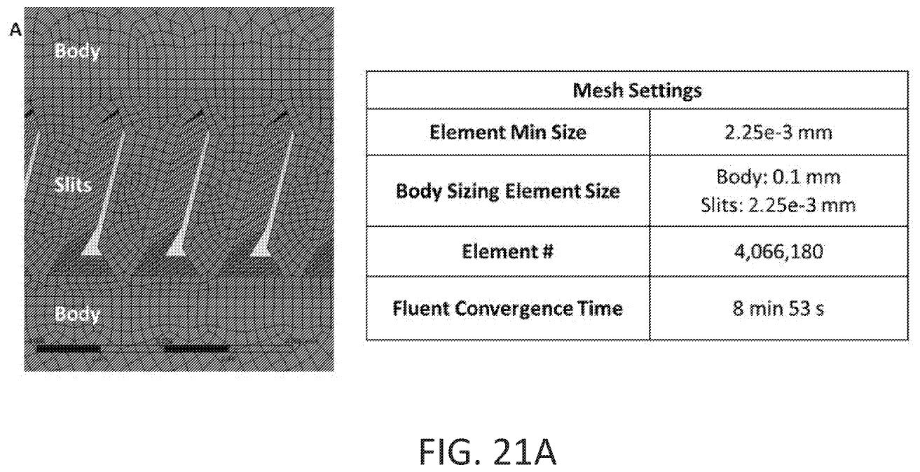

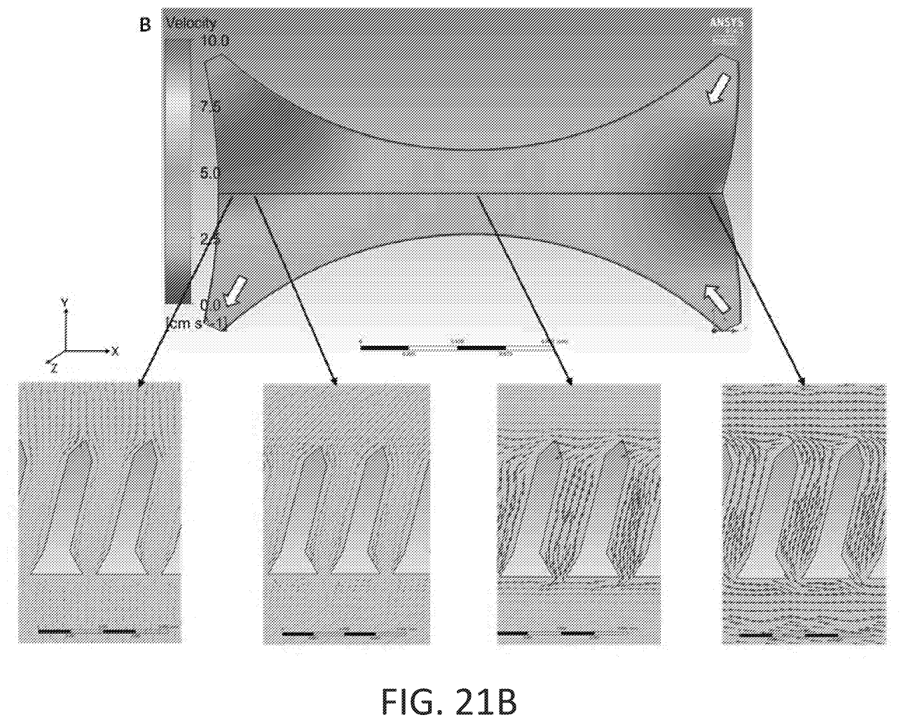

Modeling of Slit Bioreactors—Nakagawa et al. First, the minimum element size was specified for the entire geometry. Next, the geometry was subdivided into two parts: Body and Slits. Using this approach, the 4-μm slits could be assigned a specific element size that could sufficiently resolve these small regions. The top and bottom chambers (Body) were assigned identical element sizes. The element size for the slits matched the minimum element size for the entire geometry. The mesh was further refined by reducing the slit's element size as part of the mesh-independence study. This approach avoids creating an excessive number of elements in the body region that are not needed, while resolving additional detail in the slit region. The final mesh-independent system and mesh settings are shown in FIG. 21A.

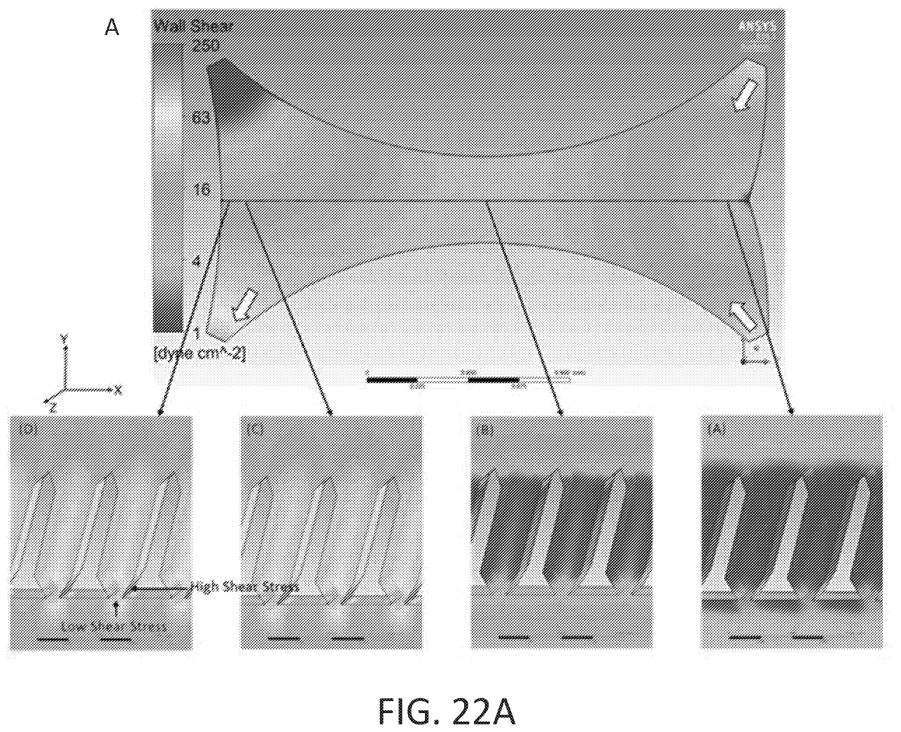

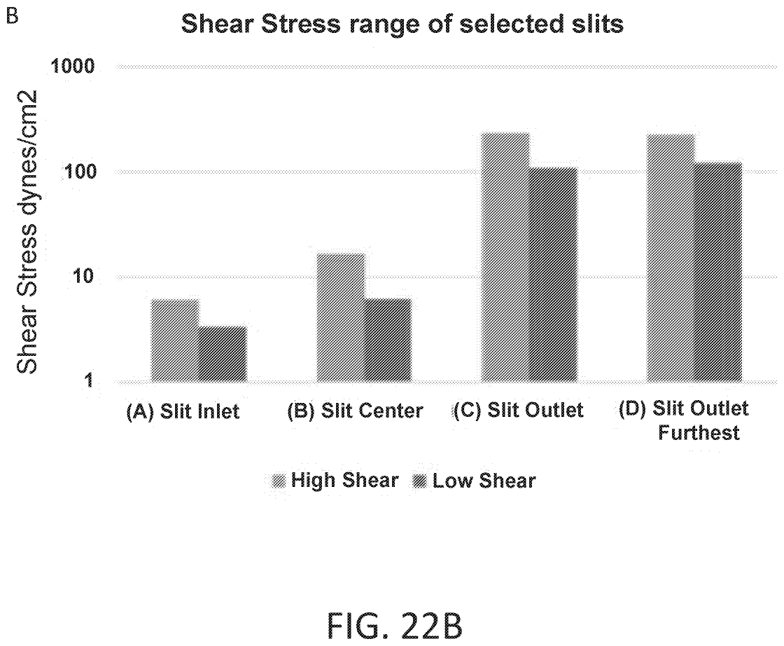

This bioreactor system contains a primary flow that pushes Mks into the 4-μm slits and a second flow that shears off proPLTs from Mks. Both flow rates are 16.7 μL/min, which corresponds to an inlet velocity of 5.85 cm/s. Velocity analysis of the Nakagawa et al. system focused on the flow patterns in the slits as well as the net flow along the length of the reactor (FIG. 21B). The velocity across the slit openings increases towards the outlet and the velocity in the upper and lower chambers is greatest at the center of the reactor. Additionally, there is predicted upward flow from the lower chamber into the upper chamber near the bioreactor inlets (FIG. 21B). The shear stress profile for the system shows an increase in shear stress towards the outlet (FIG. 22). This analysis shows that the bioreactor contains a non-uniform shear and flow environment, as well as the potential for reverse flow (from the lower to the upper chamber).

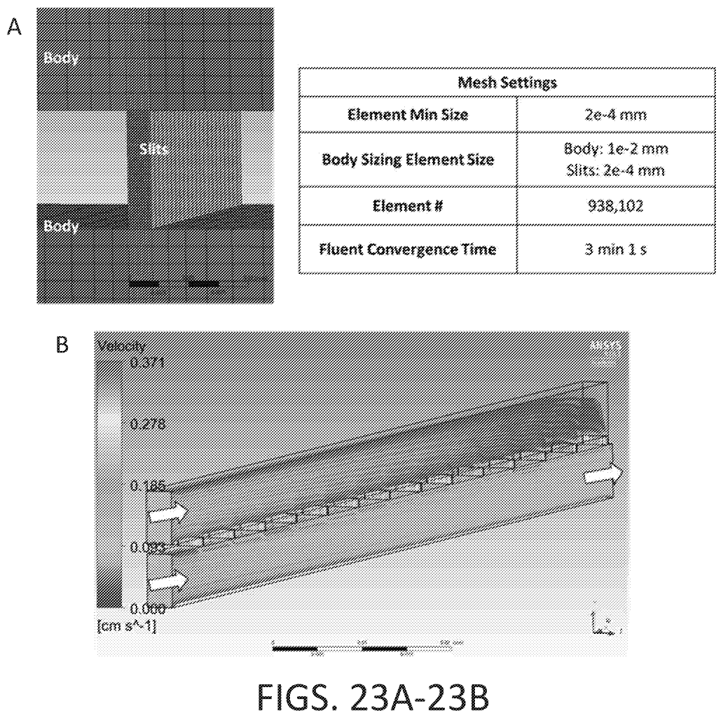

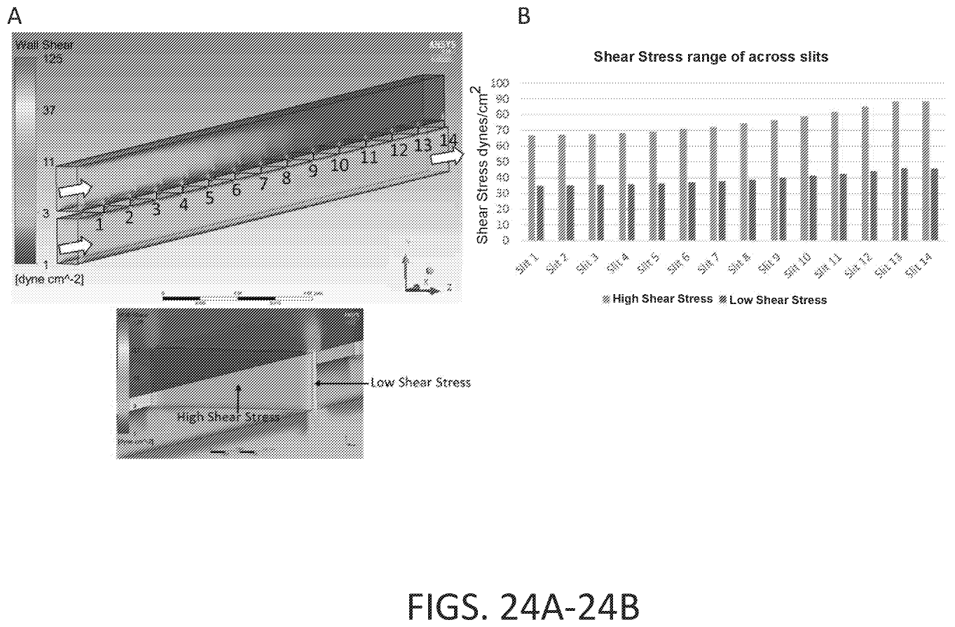

Modeling of Slit Bioreactors—Thon et al. Similar to the approach mentioned for Nakagawa et al., the distribution of the elements in the computational mesh for the Thon et al. system was refined in the region of the slits and coarser in the parallel flow channels. The mesh was further refined by sequentially reducing the element size in the slits. The final mesh-independent system and mesh settings are shown in FIG. 23. The 2-μm slits connect parallel channels where the inlet flow rate for each channel was set to 6.25 μL/h (total combined inlet flow rate 12.5 μL/h) corresponding to an inlet velocity per channel of 0.965 cm/s. Velocity analysis of the Thon et al. system focused on the slits and along the length of the reactor (FIG. 23B). The velocity across the slits within the bioreactor increases towards the outlet (FIG. 23B). The model predicts that the lower channel has a shear stress range from 3 to 6 dynes/cm² and that there is an increase in shear stress across the slits towards the end of the bioreactor (FIG. 24).

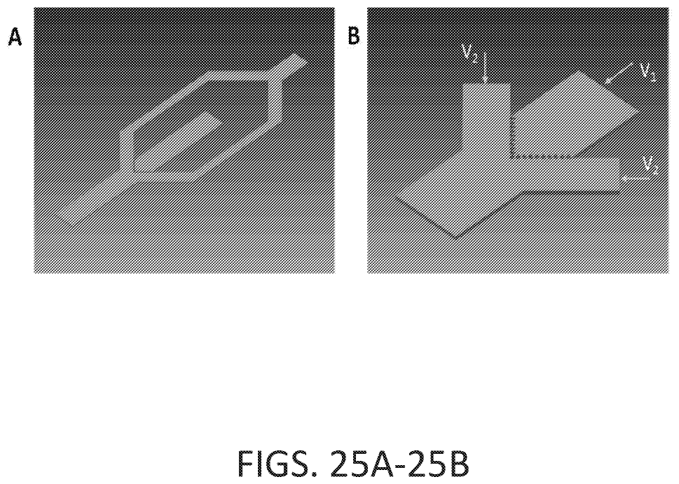

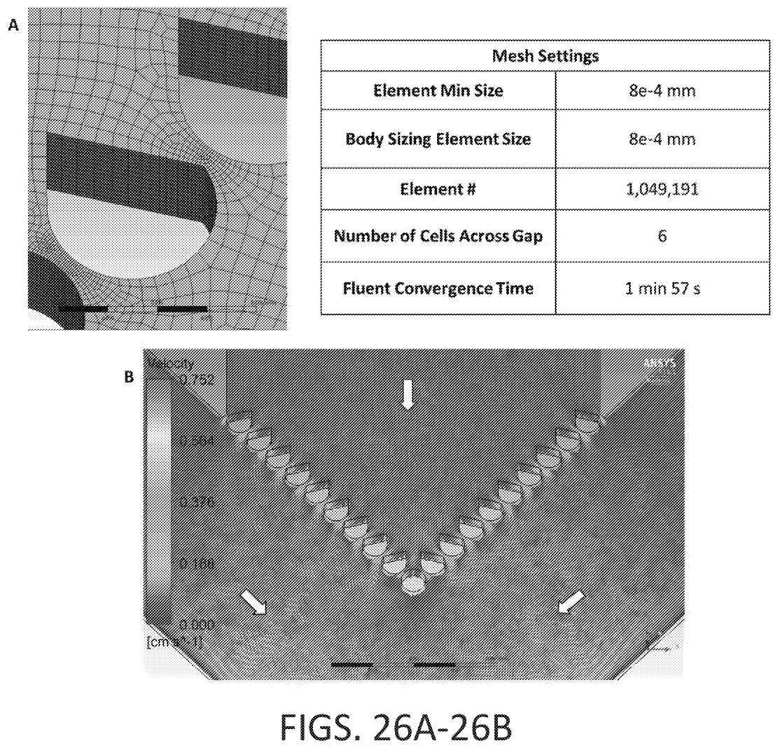

Modeling of Slit Bioreactors—Uniform-shear rate bioreactor (USRB-7 μm). Initially, the whole system, as shown in FIG. 25A was cropped to only focus on the slit region (FIG. 25B). This approach minimized the number of elements needed to analyze the system since the cropped-out regions are simple straight channels, and allowed for refinement to occur mainly in the slit region. Unlike the method presented for Nakagawa et al. and Thon et al., the bioreactor was not separated into regions—Body and Slits. Rather, the proximity method in the ANSYS meshing software was used to increase the mesh resolution in the slits. The proximity method can be used to set the minimum number of element layers within gaps (e.g., slits in the bioreactor). Thus, the slits, separated by columns, can be further refined. The minimum element size and proximity size can be specified. The minimum number of element layers (number of cells across gap) further subdivides the mesh in tight regions. The number of cells across each gap was increased along with a reduction in the minimum element size, to further refine the mesh. The final mesh-independent system and mesh settings are shown in FIG. 26A.

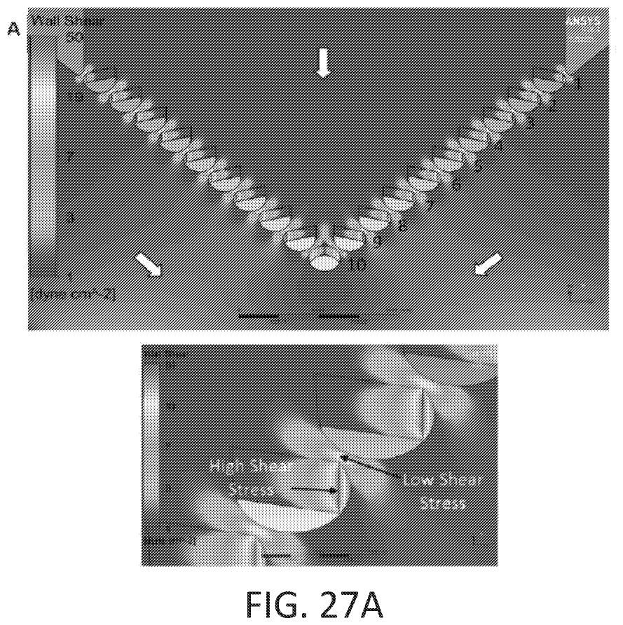

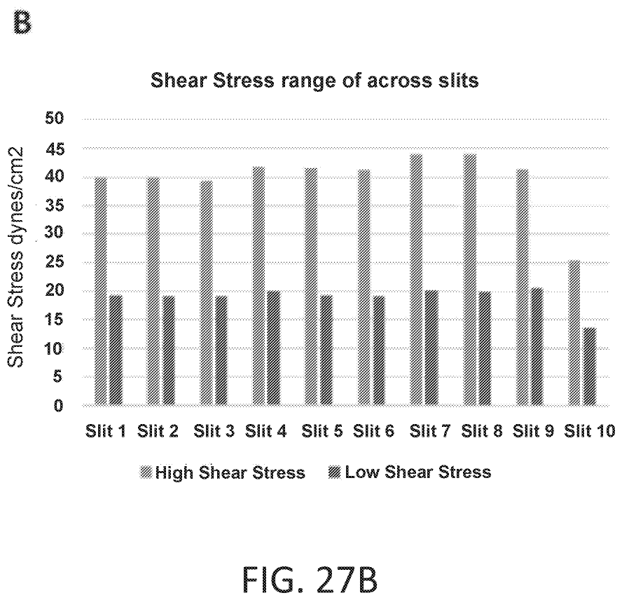

The bioreactor system contains a primary flow down the center channel that pushes Mks into the 7-μm slits and an outer channel flow that shears off proPLTs from Mks. The center channel flow rate and the combined outer channel flow rate are both 1.5 μL/min. Since the geometry was cropped, the input velocity of the center channel is set to 0.0694 cm/s (V₁ in FIG. 25B) and the outer channel to 0.0625 cm/s (V₂ in FIG. 25B). The velocity analysis of the USRB-7 μm focused around the slits (FIG. 26B). The range of shear stresses across the slits is shown in FIG. 27. Only half of the slits are presented since there is symmetry across the reactor and the other slits would have similar shear stress ranges. The lower velocity at the two slits in the center of the reactor—at the confluence of the outer channels—leads to a lower shear stress (FIG. 27—slit 10). The shear stress profile across the slits is uniform except for the slit at the center (Slit 10). However, unlike the Thon et al. and Nakagawa et al. systems, there isn't a gradual increase in shear across the slits along the length of the bioreactor.

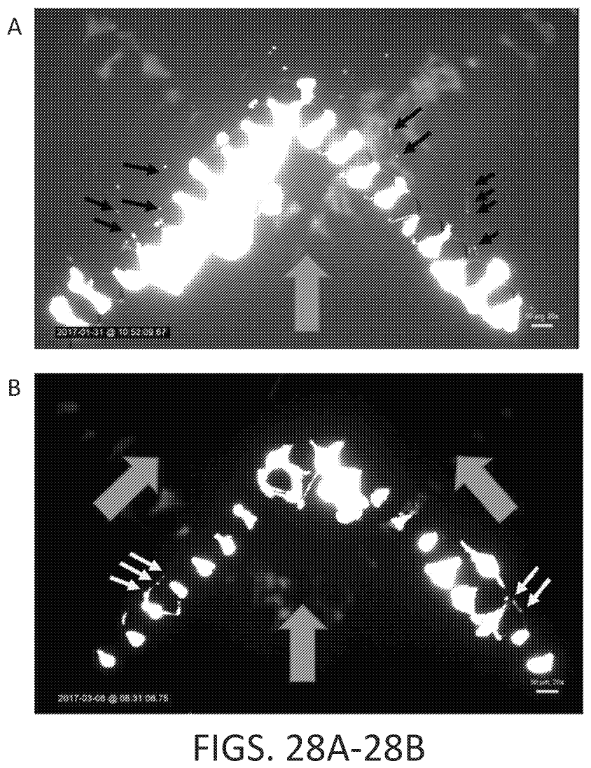

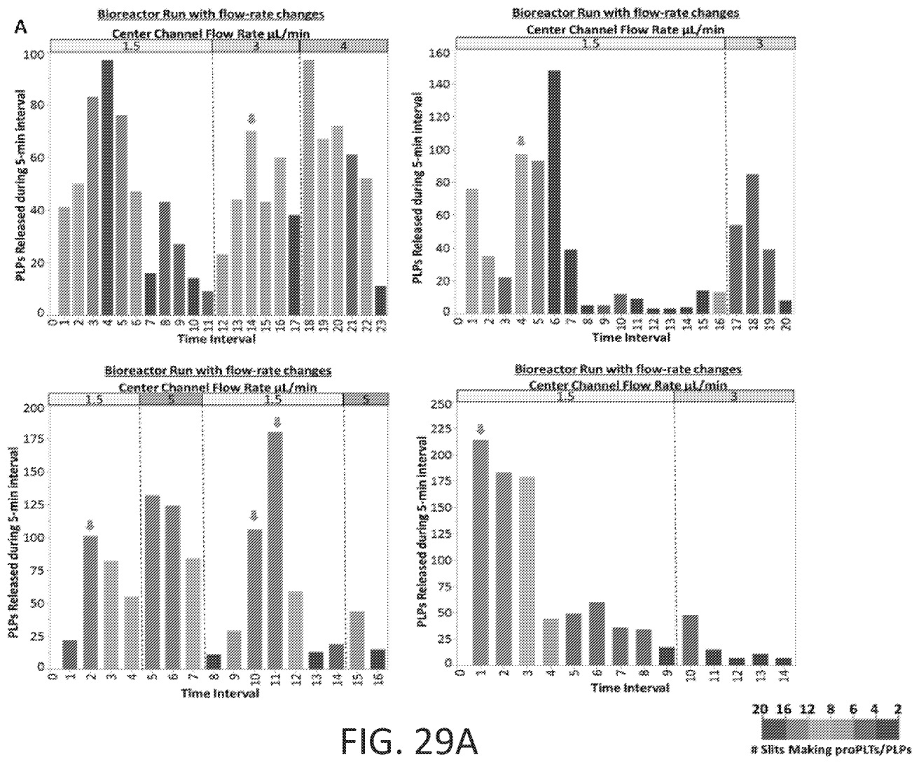

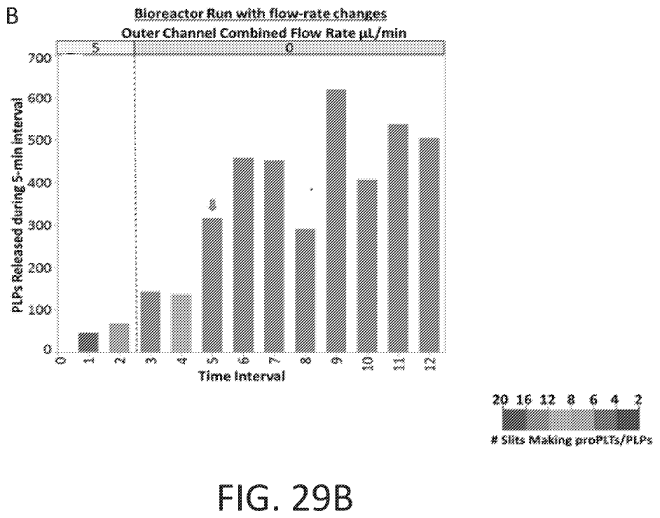

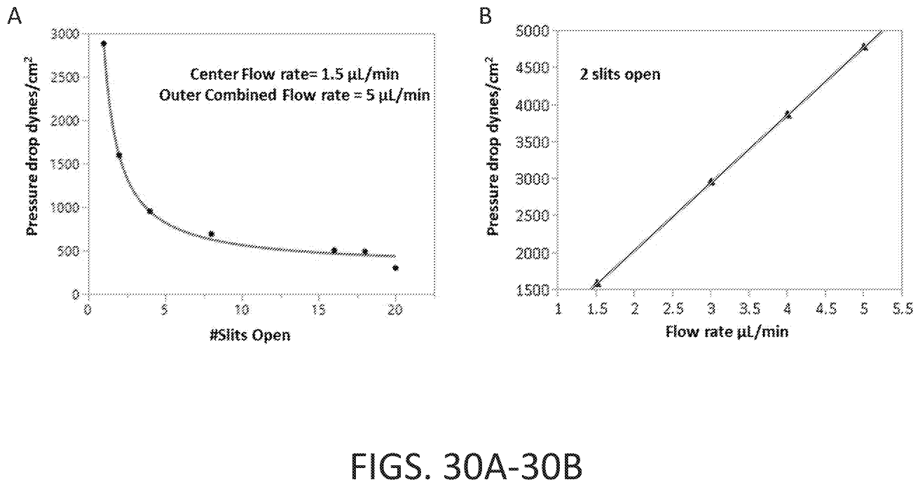

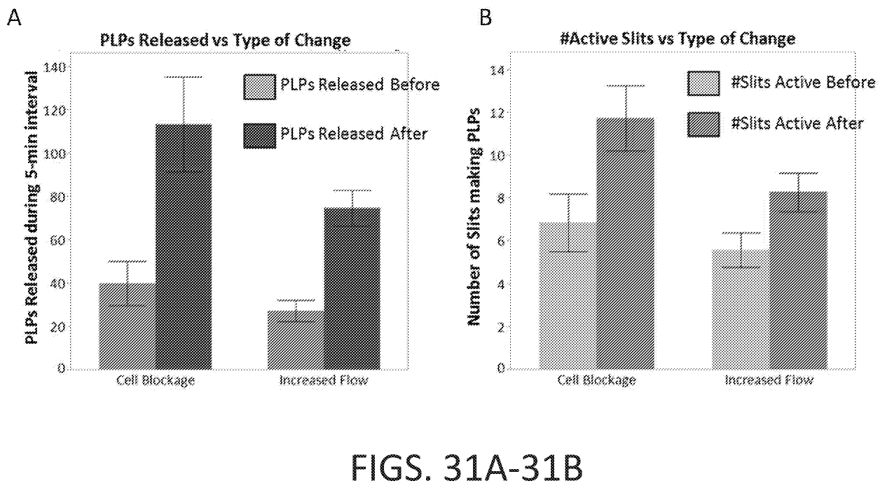

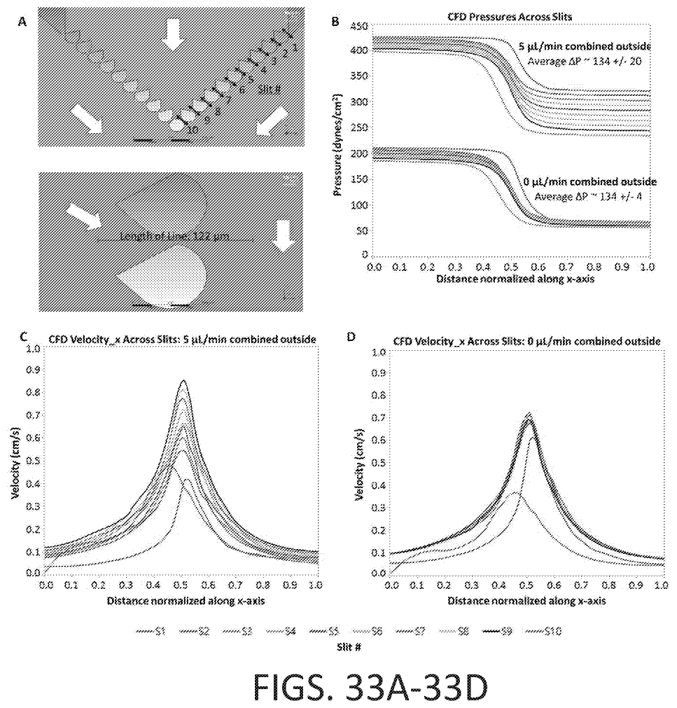

PLP Quantification. Platelet-like-Particles (PLPs) were counted on proPLTs based on the number of “beads” observed (FIG. 28). Profile of PLPs release per 5-min interval for various reactors show the response of the system after a blockage event and after a change in flow rate in the center channel (FIG. 29A) or outer channel flow rate change (FIG. 29B). The pressure drops across the bioreactor when a slit is completely blocked or when a center channel flow rate change occurs showed a gradual CFD pressure increase (FIG. 30). Additionally, analyzing the experimental videos collected showed that a slit-blockage or center channel flow rate change led to an immediate increase in PLPs (FIG. 31A) and an increase in the number of slits that were actively making PLPs (FIG. 31B). Individual slits within the USRB-7 μm were analyzed with CFD along the center height (z=20 μm). The slits were aligned along the x-axis and a line was created in the CFD output in each slit (FIG. 33A). CFD data, such as pressure (FIG. 33B), and velocity (FIG. 33C-33D), was extracted for individual slits.

Bioreactor Fabrication. Briefly, a chrome mask (Front Range Photomask, Palmer Lake, Colo.) of the USRB-7 μm was used to create a master silicon wafer (WRS Materials, San Jose, Calif.) on which polydimethyl siloxane (PDMS) solution (Slygard 184 Kit; Electron Microscopy Sciences, Hatfield, Pa.) was poured to cast a mold.

A 2D design of the bioreactor was created in AutoCAD 2014 (San Rafael, Calif.) and then printed onto a chrome mask (Front Range Photomask, Palmer Lake, Colo.). A silicon wafer (WRS Materials, San Jose, Calif.) was spin-coated with SU8-2035 photoresist (MicroChem Corp, Westborough, Mass.) at 4000 RPM for 30 s to achieve a photoresist height of 40 μm. The wafer was soft-baked at 65° C. for 3 min and then hard-baked at 95° C. for 6 min. Afterwards, the wafer was exposed to UV light for 17 s using a Karl Suss MA6 Mask Aligner (SUSS MicroTec, Garching, Germany). The exposed resist was then baked at 95° C. for 6 min. Finally, the resist was developed using SU8 developer solution (MicroChem) for 2 min and dried with a nitrogen gun. The dry wafer was silanized overnight (5 μL of 1H,1H,2H,2H-perfluorooctyltrichlorosilane; Alfa Aesar, Ward Hill, Mass.) in a vacuum chamber. Next, a 1:10 curing agent to polydimethyl siloxane (PDMS) solution (Slygard 184 Kit, Electron Microscopy Sciences, Hatfield, Pa.) was poured over the wafer to cast a mold that was placed in an oven at 65° C. overnight. The PDMS mold was then cut, holes for inlets and outlets created with a 2-mm punch, and the PDMS plasma-bonded (Model BD-20; Electro-Technic Products, INC, Chicago, Ill.) to an ethanol-cleaned premium plain glass slide (25×75×1 mm; VWR, Radnor, Pa.).

Cell Culture. Unless otherwise specified, all reagents were obtained from Sigma-Aldrich (St. Louis, Mo.), and cytokines from Peprotech (Rocky Hill, N.J.). Media viscosity used in the CFD simulations was measured using a Cannon-Fenkse Routine Viscometer—size 50 (Cannon Instrument Co., State College, Pa.). Two measurements were conducted for IMDM, IMDM+10% FBS, and IMDM+20% BIT, at 37° C. in a water bath. Previously frozen mobilized peripheral blood (mPB) CD34⁺ cells from the Fred Hutchinson Cancer Research Center (Seattle, Wash.) with Northwestern University Institutional Review Board approval were grown in 78% IMDM (Gibco, Carlsbad, Calif.), 20% BIT 9500 Serum Substitute (STEMCELL, Vancouver, BC, Canada), 1% Glutamax (Gibco), 1 μg/mL low-density lipoproteins (Calbiochem, Whitehouse Station, N.J.), 100 U/mL Pen/Strep, 100 ng/mL TPO, 100 ng/mL SCF, 10 ng/mL IL-6, 10 ng/mL IL-11, and 2.5 ng/mL IL-3 (R&D Systems, Inc., Minneapolis, Minn.). Cells were maintained between 100,000 to 400,000 cells/mL at 37° C., 5% CO₂, and 5% O₂ for 5 days (Panasonic incubator MCO-170M, Wood Dale, Ill.). On day 5, the cytokines were replaced with 100 ng/mL TPO, 100 ng/mL SCF, 10 ng/mL IL-9, 10 ng/mL IL-11, and 10 ng/mL IL-3. Cells were maintained at a density of 250,000 to 500,000 cells/mL and kept at 37° C., 5% CO₂, and 20% O₂ until day 7. On day 7, cells were selected using anti-CD61-conjugated magnetic microbeads (Miltenyi Biotech Inc, San Diego, Calif.) and then cultured in medium with 100 ng/mL TPO, 100 ng/mL SCF and 6.25 mM nicotinamide thereafter. The cells were maintained at a density between 250,000 to 500,000 cell s/mL and kept at 37° C., 5% CO₂, and 20% O₂.

Bioreactor Perfusion with Mks. The USRB-7 μm was positioned on a Lumascope microscope v500 (Etaluma Inc., Carlsbad, Calif.) placed inside an incubator (Thermo Scientific, Waltham, Mass.) maintained at 37° C. and 5% CO₂. Separate syringe pumps (NE-300, New Era. Pump Systems Inc., Farmingdale, N.Y.) were used for each flow channel. A 5-mL glass syringe (81520, Hamilton Company, Reno, Nev.) was used for the outer channels and a 2.5-mL glass syringe (81420, Hamilton) was used for the center channel. Media (78% IMDM (Gibco, Carlsbad, Calif.), 20% BIT 9500 Serum Substitute (STEMCELL, Vancouver, BC, Canada), 1% Glutamax (Gibco), 1 mg/mL low-density lipoproteins (Calbiochem, Whitehouse Station, N.J.), 100 U/mL Pen/Strep) without cytokines was perfused throughout the bioreactor for 30 min at 6.5 μL/min prior to Mk introduction. On day 10, 11, or 12 of Mk culture, Mks at density of 50,000/mL were stained for 15 min with 1 μM Calcein AM at 37° C. After the 30-min media perfusion, 25,000 Mks (a sufficient number to observe the system dynamics and how often they might repeat and under what conditions, without clogging the slits) were microinjected into the tubing upstream from the reactor. No Mks were present within the syringes. A video was recorded of each bioreactor for 1-2 h.

Video Analysis. Videos (6 frames-per-second) were recorded for each experimental run using the Lumascope v500, equipped with High Sensitivity Monochrome CMOS Sensor camera, using a 20× or 40× objective. Each video was analyzed for every 5-min time interval for the duration of an experiment. One half of the bioreactor (10 slits) was analyzed at one time throughout 5-min time intervals for the entire video recorded. This process was repeated on the other half of the reactor. The data from each half of the reactor was then combined for each 5-min time interval. During each interval, only proPLTs and PLPs that originated from trapped. Mks in the slits were counted. Additionally, for some videos, pre-released particles flowing into and out of the slits were counted separately. Mks can give rise to particles without shear and these could be present in the suspension that was microinjected into the system. To increase accuracy, the videos were played at a slower speed during times of high PLP release activity. The 5-min interval was selected because it allowed effective analysis and perturbation of the dynamics of the process. Pre-staining Mks with Calcein AM allowed the Mks trapped in the reactor, as well as proPLTs/PLPs, to be clearly observed.

The following videos are part of the present disclosure and are available upon request:

Video 1: proPLT Formation. Trapped Mk extending proPLTs through slit. Flow in the outer channels applies shear on the extensions further elongating them leading to fragmentation after several minutes. Time units: h:min:s. Scale bar=50 μm. Flow rate of 1.5 μL/min in the center channel with 5 μL/min in the combined outer channels.

Video 2: Rapid PLPs releases. Trapped Mks in a slit, rapidly releasing many individual PLPs in seconds. Time units: h:min:s. Scale bar=35 μm. Flow rate of 1.5 μL/min in the center channel with 5 μL/min in the combined outer channels.

Video 3: Major slit-blockage by Mks. Upon cell-capture at a slit, there is a noticeable increase in proPLT/PLP activity across the bioreactor, Scale bar=50 μm. Flow rate of 1.5 μL/min in the center channel with 5 μL/min in the combined outer channels.

Video 4: Impact of turning off outside flow rate. Starting with an outside flow rate at 5 μL/min, Mks observed making proPLTs/PLPs. After turning off the outside flow rate, there is an increase in proPLT/PLP productivity across the bioreactor. Scale bar=50 μm. Flow rate of 1.5 μL/min in the center channel.

Video 5: Rapid proPLT formation and PLP releases. Mks are trapped and rapidly converted to proPLTs/PLPs at the slits. Scale bar=50 μm. Flow rate of 1.5 μL/min in the center channel with 0 μL/min in the combined outer channels.

Statistical Analysis. JMP Pro 11 (SAS Institute Inc., Cary, N.C.) was used to generate histograms, distributions, and standard errors of the video analysis data for released proPLTs/PLPs.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### CFD Modeling of Shear Rates Within Slit Bioreactors

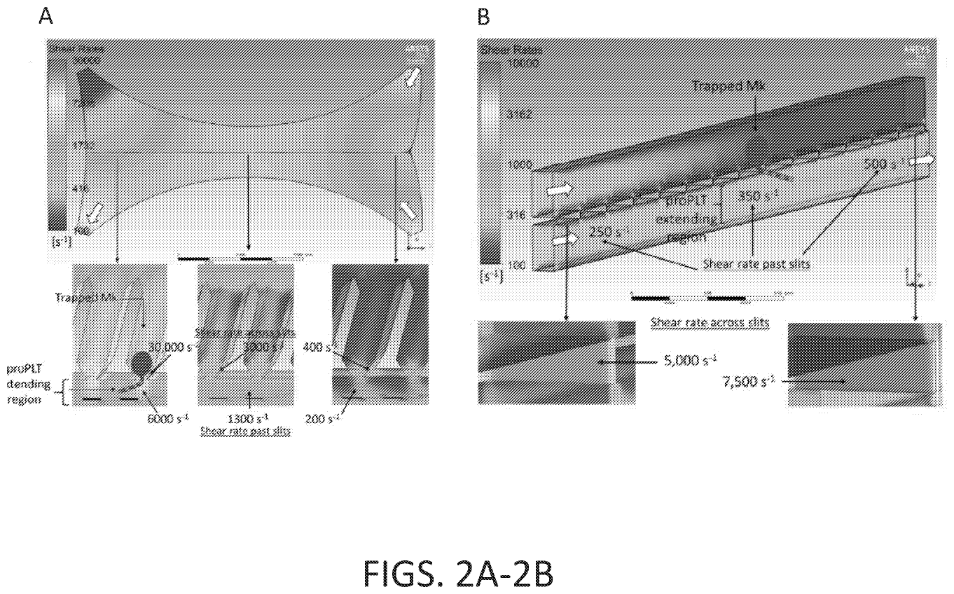

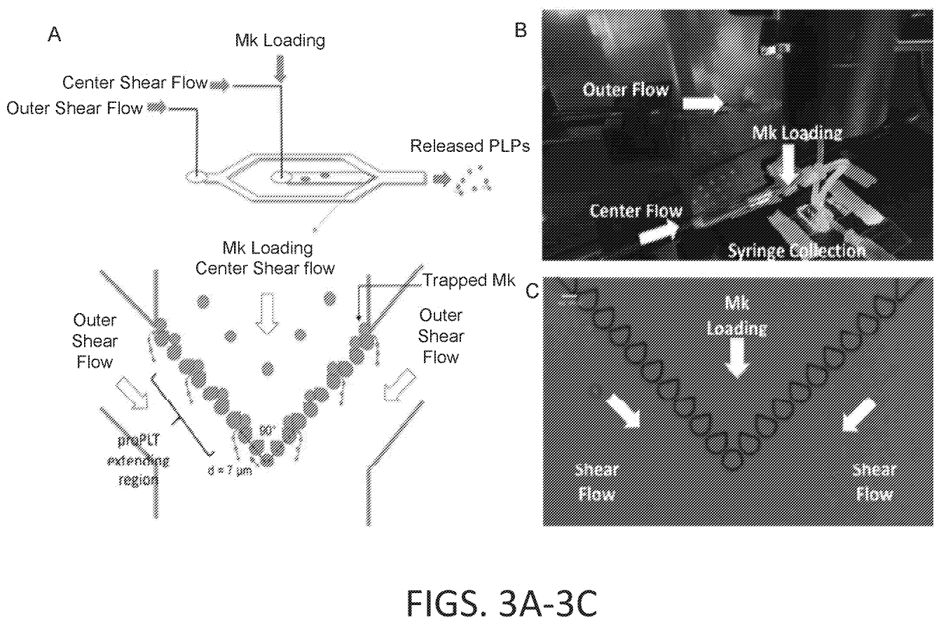

Computation fluid dynamics (CFD) was used to evaluate the flow and shear conditions within several slit bioreactors (Table 1), CFD analysis of the Nakagawa bioreactor (FIG. 21), which uses 4-μm slits predicts slit shear rate that ranges from 400 s⁻¹ near the inlet to 30,000 s⁻¹ near the outlet (FIG. 2A). The calculated shear rate on proPLT extensions in the lower chamber ranges from 200 s⁻¹ near the inlet up to 6,000 s⁻¹ near the outlet (FIG. 2A). Additionally, simulations predict a net flow from the lower chamber into the upper chamber near the bioreactor inlets (FIG. 21B). Based on this CFD analysis, the flow and shear environment varies significantly across the bioreactor (FIG. 2A, FIGS. 21B, and FIG. 22). Thon et al. analyzed their 2-μm slit reactor using CFD. CFD analysis of this system is provided herein (FIG. 23). Consistent with Thon et al., simulations described herein showed that the shear rate in the slits increases from the inlet toward the outlet of the bioreactor. The calculated shear rate in the open slits ranges from 5,000 s⁻¹ at the inlet to 7,500 s⁻¹ at the outlet (FIG. 2B). The calculated shear rate below the slits, along the lower channel wall, ranges from 250 s⁻¹ near the inlet to 500 s⁻¹ at the outlet (FIG. 2B), similar to that reported by Thon et al. This reactor provides a more uniform shear profile compared to that of Nakagawa et al. However, there is still an increase in slit shear rates toward the bioreactor outlet (FIG. 2B and FIG. 24).

### Example 2

### CFD-Driven Design and Assessment of a Uniform-Shear-Rate Bioreactor

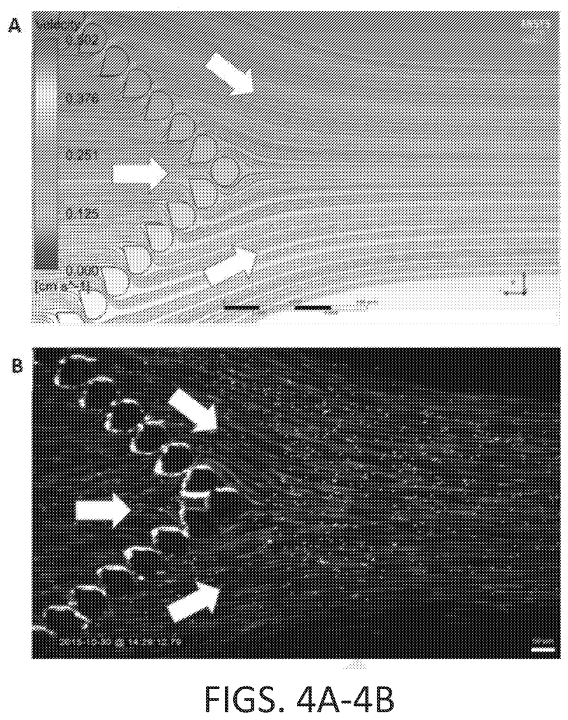

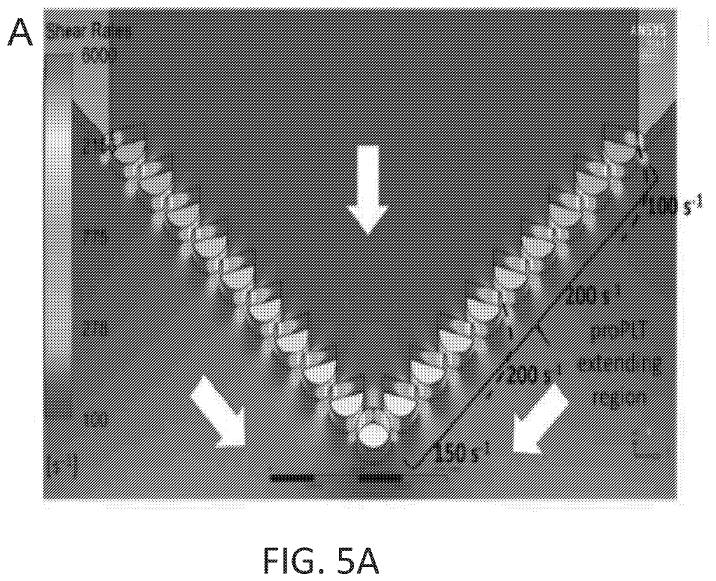

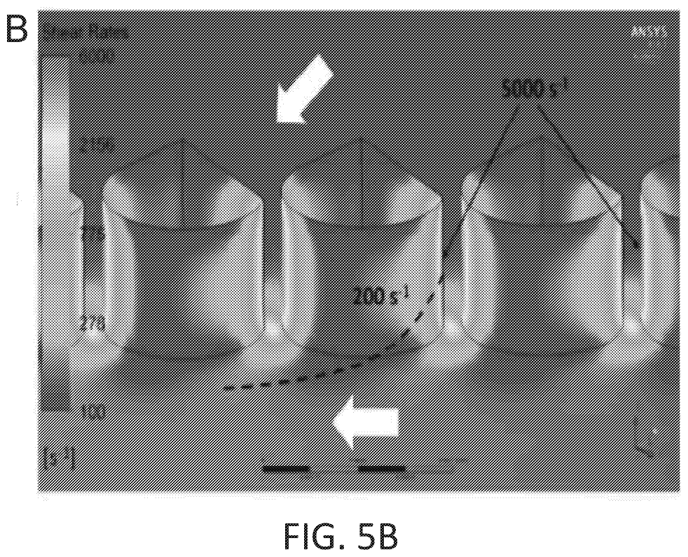

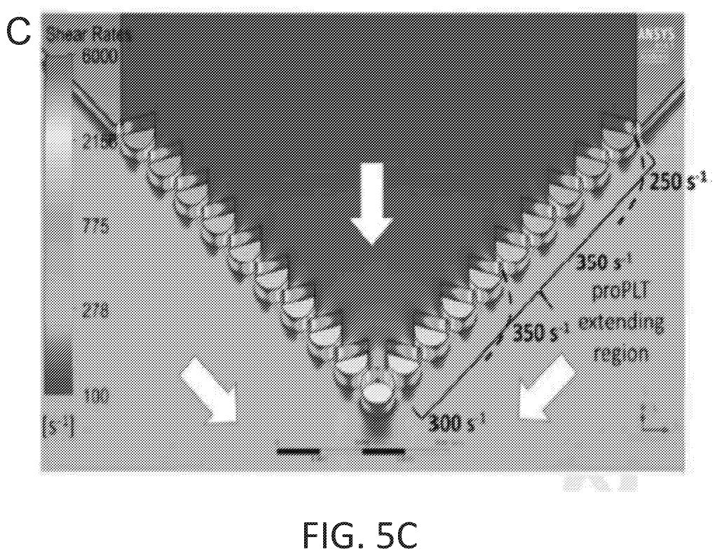

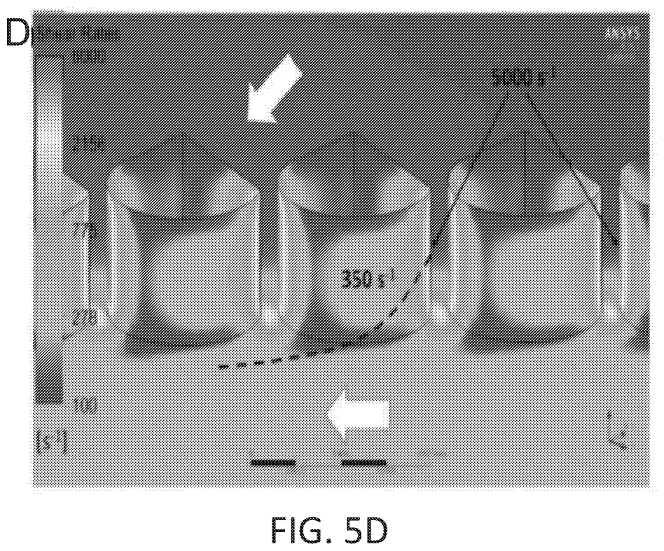

A thorough CFD analysis was conducted on potential new slit bioreactor designs to avoid the CFD-predicted nonuniform flow and shear profiles of current slit bioreactors. In the optimized bioreactor system of the present disclosure, Mks enter a center channel where a V-shaped array of twenty 7-μm slits separates the Mks from outside flows converging at 90° C. (FIG. 3, FIG. 25 and FIG. 26). The slit dimension was chosen to retain mature Mks—usually >20 μm—and to prevent large pressure drops and flow stagnation. The height of the bioreactor was chosen to be 40 μm, similar to blood sinusoid dimensions. To experimentally visualize and confirm flow patterns in the system, 1-μm fluorescent beads were used to map the streamlines of the cell-free system, and showed good agreement with the CFD streamlines (FIG. 4). Uniform shear profiles across and downstream of the slits were confirmed through CFD simulations of the cell-free system. For center channel and combined outer channel flow rates of 1.5 μL/min each, the 7-μm slits have a maximum calculated shear rate of 5,000 s⁻¹, except for a maximum shear rate of 2,800 s⁻¹ for the two slits at the end of the V where the flow in the outer channels converges (FIGS. 5A-5B and FIG. 27). ProPLTs extending through the slits would experience a shear rate range from 100 to 200 s⁻¹ (past the slits in the open channel) (FIGS, 5A-5B). Increasing the combined outer channel flow rate to 5 μL/min did not affect the shear rate through the slits (maximum remained 5,000 s⁻¹), but the shear rate that would be experienced by proPLTs increased to 250-350 s⁻¹ (FIGS. 5C-5D). Therefore, the USRB-7 μm allows Mks trapped at the slits and extending proPLTs to experience similar shear rates regardless of location within the bioreactor.

### Example 3

### Understanding Shear Forces in the Presence of Cell Blockages

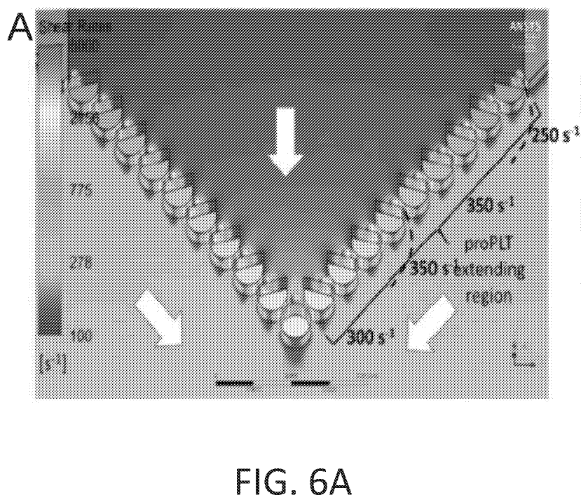

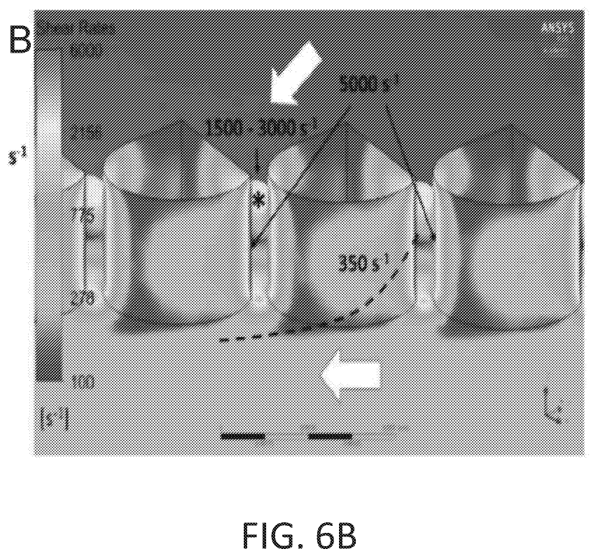

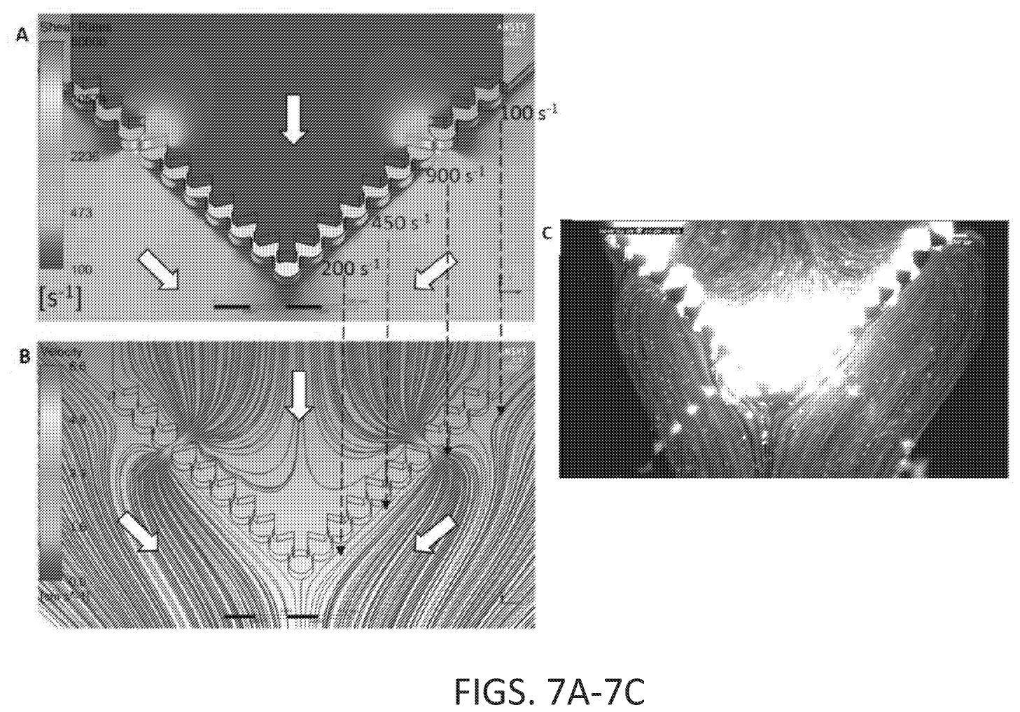

Cell-blockage scenarios for the slits were simulated using CFD, with a center channel flow rate of 1.5 μL/min and a combined outer channel flow rate of 5 μL/min. First, 20-μm sized spheres were modeled just upstream of slits (i.e., partial blockage). The simulations predict that these cells would experience a shear rate of 1,500-3,000 s⁻¹ (FIG. 6). Second, the system was modeled with only 2 open slits by completely blocking the remaining 18 slits. ProPLTs extending past the slits are expected to experience a shear rate of 100-900 s⁻¹ (FIG. 7A). The shear rate is the highest (900 s⁻¹) near the 2 open slits where the velocity is the highest (FIGS. 7A-7B). The lowest shear rate (100 s⁻¹) occurs upstream of the open slits where it appears that the flow from the open slit is re-directing the outside flow away from the slits (FIGS. 7A-7B). Similarity between the simulation streamlines and experimental streamlines was confirmed using cells and 1-μm fluorescent beads (FIG. 7B vs. FIG. 7C). Thus, CFD can help understand the flow profile, as well as estimate the shear rates, in the USRB-7 μm when Mks are trapped at the slits.

### Example 4

### Uniform-Shear-Rate Bioreactors Promote proPLT and Rapid PLP Generation From Mks

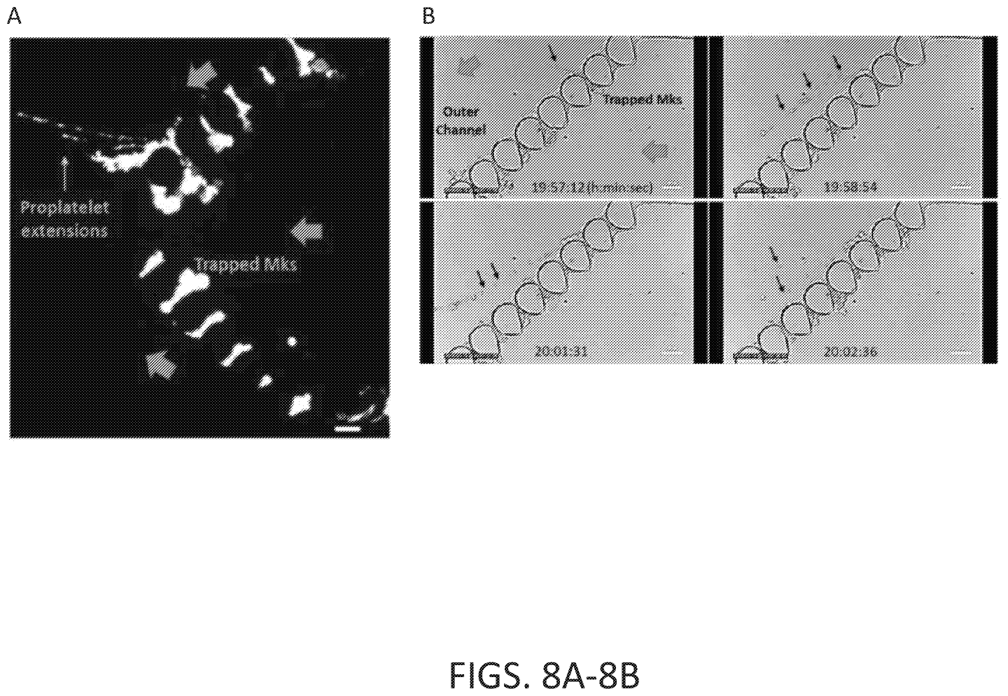

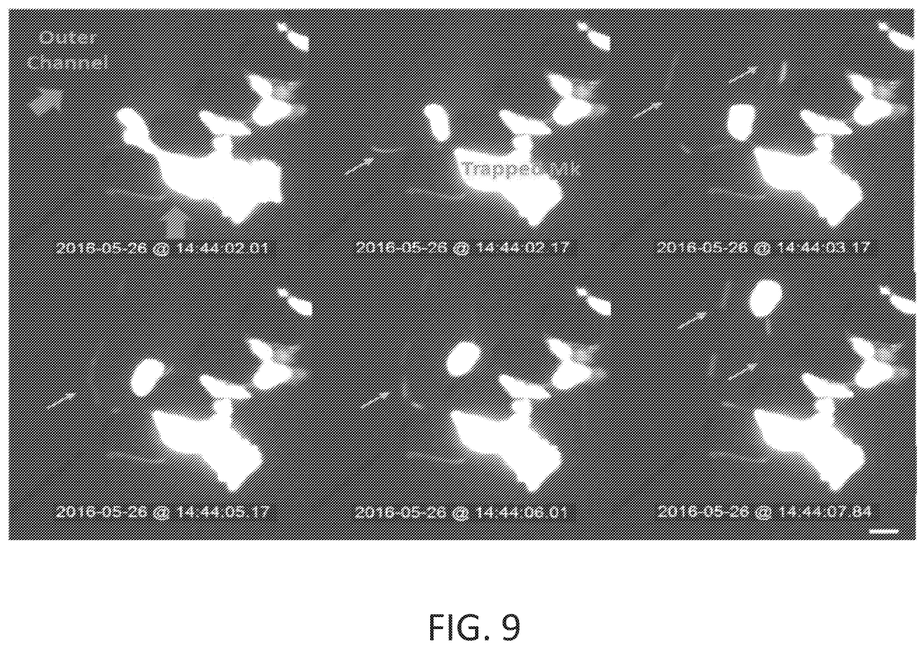

After design and fabrication of the USRB-7 μm (FIG. 3), and validation of the flow patterns (FIG. 4 and FIG. 7C), the capability of the system to promote proPLT formation from mobilized peripheral blood (mPB)-derived Mks was assessed. Experiments showed that, depending on the size of the trapped Mks, 1-3 cells can occupy a slit. Importantly, the shear environment within the USRB-7 μm could stimulate proPLT formation from trapped Mks (FIG. 8). Mks were stained with Calcein to allow clearer visualization of the proPLT formation process (FIG. 8A). Trapped Mks within the slits extruded their bodies and elongated into the characteristic proPLTs with beads-on-a-string morphology (FIG. 8B; Video 1). Interestingly, the USRB-7 μm microenvironment also promoted trapped Mks to release individual PLPs directly from their bodies. Some Mks, immediately after slit capture, rapidly released dozens of PLPs within seconds (FIG. 9; Video 2).

Example 5

### Evaluating PLP-Release Kinetics Under Different Flow Conditions

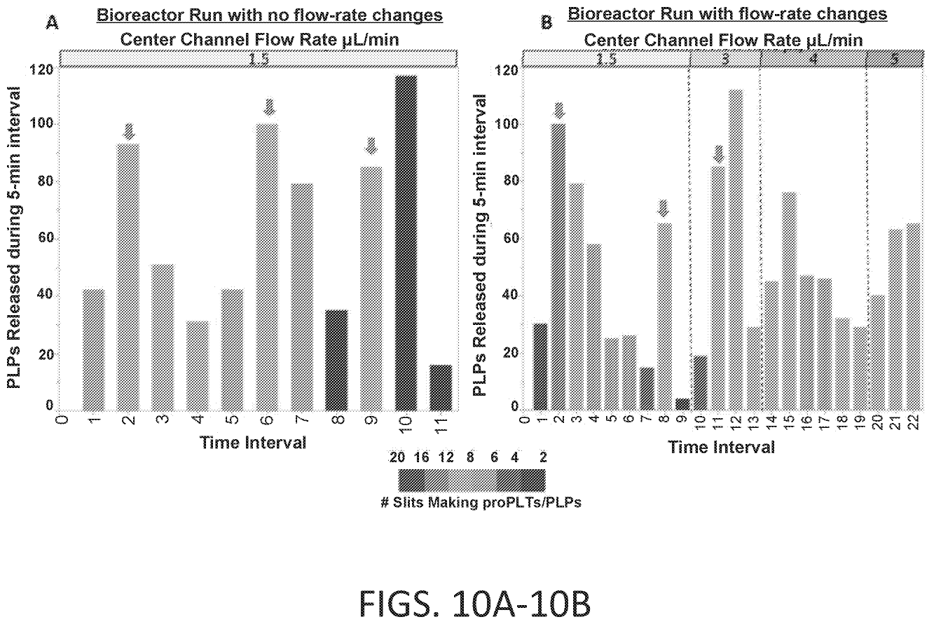

The kinetics of the USRB-7 μm were analyzed to identify conditions that change Mk behavior by counting the number of Calcein-stained PLPs that originate from Mks trapped at the slits per 5-min time interval across individual experimental runs (FIG. 10). The number of PLPs-on-a string that were observed on proPLTs were also counted as released PLPs (FIG. 28) and it was estimated that approximately 30% of PLPs released were from proPLTs. It was observed that, when an incoming Mk blocked the flow of an open slit, proPLT/PLP formation and the number of slits making PLPs greatly increased within the bioreactor (FIG. 10A—green arrows; Video 3). The blockage most likely increased the pressure drop across the slits, thus, trapped Mks were exposed to an immediate higher pressure and shear that increased their productivity. This observation led to the hypothesis that introducing a step-increase in the center channel flow rate may mimic the effects of cell-blockage. Indeed, similar responses after multiple step-increases in flow rate were observed during five separate experimental runs (FIG. 10B and FIG. 29A). The calculated CFD pressure drop across the slits increased continuously as more slits were occupied, especially when few slits remained open (FIG. 30A). Further, increasing the flow rate of the system with a constant number of open slits increased the calculated CFD pressure drop across the slits in a linear manner (FIG. 30B). The experimental observations and CFD analysis support the hypothesis that a pressure drop increase by a blockage event or flow rate change could increase Mk productivity, as both types of changes increased the immediate number of PLPs released by approximately three-fold and the number of active slits by 30-50% (FIG. 31).

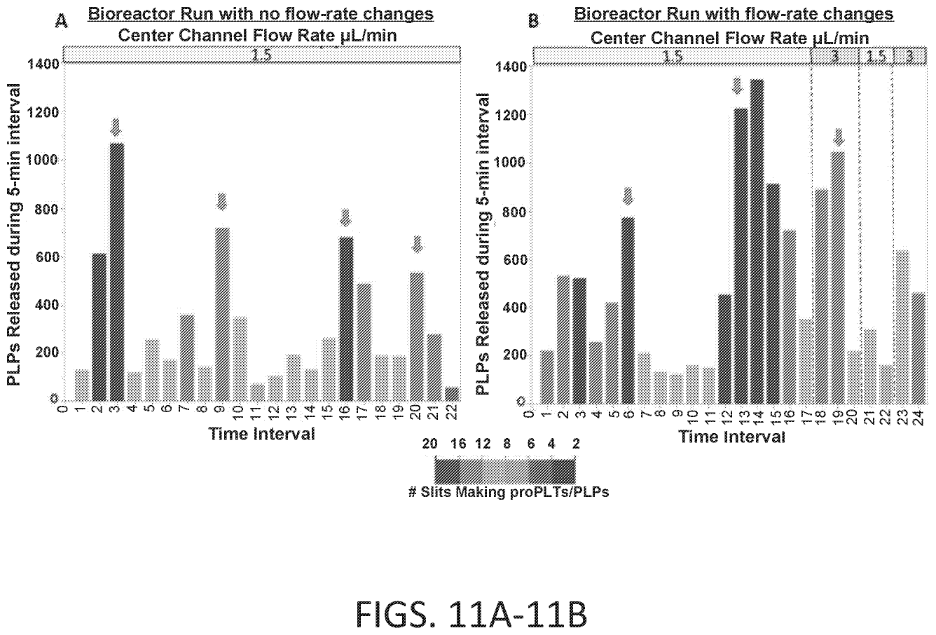

While trying to remove a small bubble from the outer channel during an experiment, the flow of the outer channels was inadvertently stopped. Surprisingly, when the outer channel flow was completely stopped, the rate of proPLT and PLP release dramatically increased (FIG. 11 vs. FIG. 10; see also FIG. 29B). The outer channel flow-rate is intended to impose shear forces on the extending proPLTs, so it is also a key parameter of the system. Yet, it was demonstrated that turning off the outer channel flow rate dramatically changed the Mk behavior and greatly increased PLP release (Video 4). Further, an increase in productivity was still observed when an incoming Mk blocked the flow of an open slit (FIG. 11A—green arrows) or by introducing a step-increase in flow rate in the center channel (FIG. 11B), similar to that seen when the outside flow was maintained at 5 μL/min (FIG. 10). Under this new operating condition, upon capture, some Mks continued to rapidly release dozens of PLPs within seconds (Video 5).

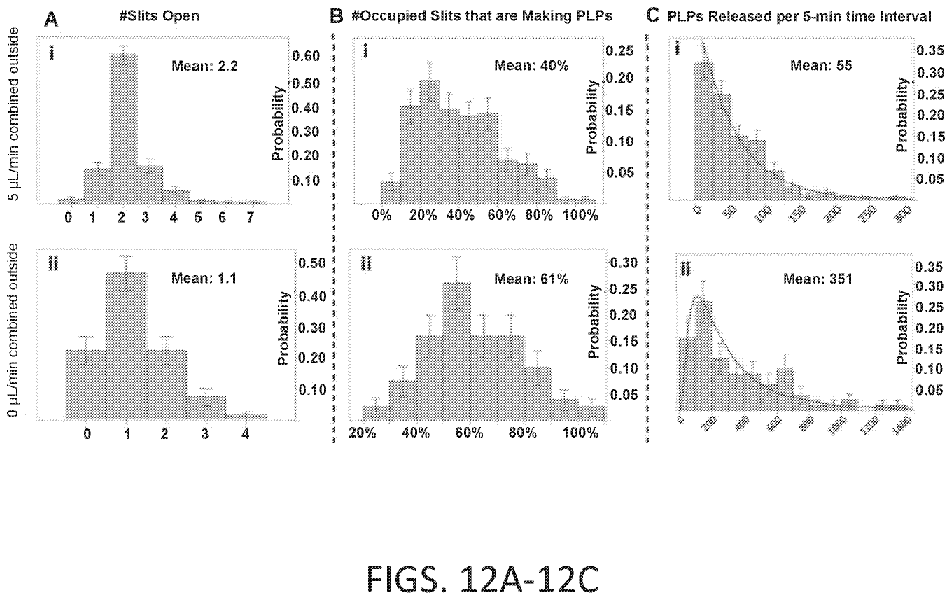

Bioreactor runs were compared using outer channel combined flow rates of 5 μL/min (9 bioreactor runs across 3 different Mk cultures) and 0 μL/min (4 bioreactor runs across 2 different Mk cultures) (FIG. 12). The average number of open slits was 2 for 5 μL/min and 1 for 0 μL/min combined outer channel flow rates (FIG. 12A). On average, 40% of the occupied slits were actively making proPLTs/PLPs under the 5 μL/min outer flow condition, whereas 61% were active when operating at 0 μL/min outer flow condition (FIG. 12B). The number of PLPs released per 5-min time interval had a mean of 55 and followed an exponential decay curve for the 5 μL/min outer flow condition, while there was a six-fold higher mean of 351 PLPs released with a log-normal distribution for 0 μL/min (FIG. 12C). Thus, unexpectedly, an outer channel flow rate of 0 μL/min greatly increased PLP production compared to 5 μL/min. An interesting observation from both environments is that the productivity increase from a blockage event (green arrow) could carry over into the next interval if the blockage occurred near the end of that interval (FIG. 10A—intervals 9 to 10, FIG. 10B—intervals 11 to 12, FIG. 11B—intervals 13 to 14, and FIG. 29B—intervals 5 to 6).

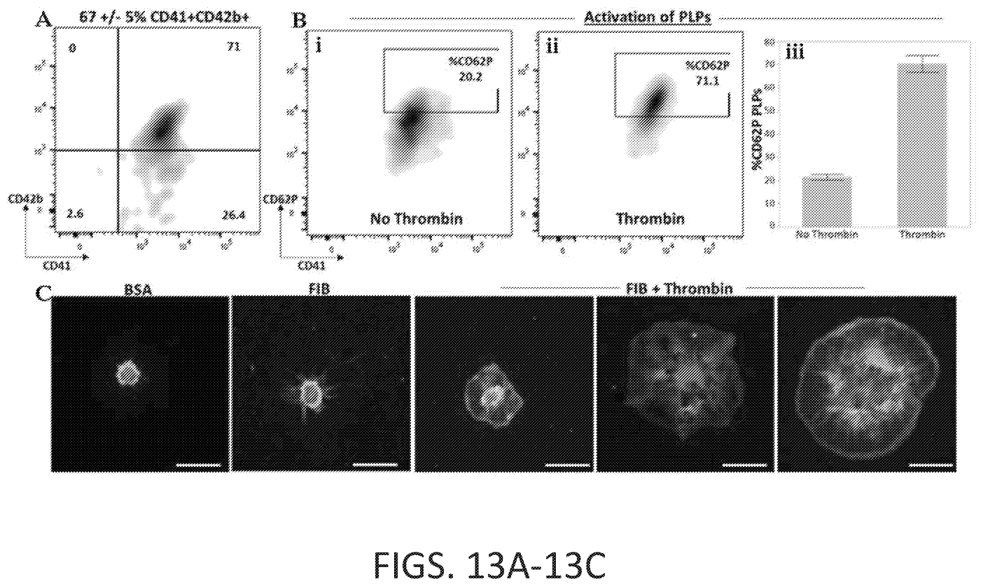

It is important to demonstrate that the PLPs produced exhibit functional activity. Due to the higher productivity, the effluent of three bioreactors operated with an outer channel combined flow rate of 0 μL/min was analyzed. Calcein⁺ PLPs were approximately 67% CD41⁺CD42b⁺ (FIG. 13A). Functional activity of CD41⁺CD42b⁺ PLPs was evaluated via expression of CD62P—a transmembrane glycoprotein that is translocated by granules to the surface of platelets after activation—both before (FIG. 13Bi) and after adding thrombin (FIG. 13Bii) to activate the PLPs. The average percentage of CD62P⁺ PLPs increased from approximately 20% to approximately 70% after thrombin addition (FIG. 13Biii). Confocal analysis of PLPs on fibrinogen revealed a characteristic tubulin ring in the absence of thrombin and highly spread PLPs in the presence of thrombin (FIG. 13C), which is similar to the behavior of fresh platelets.

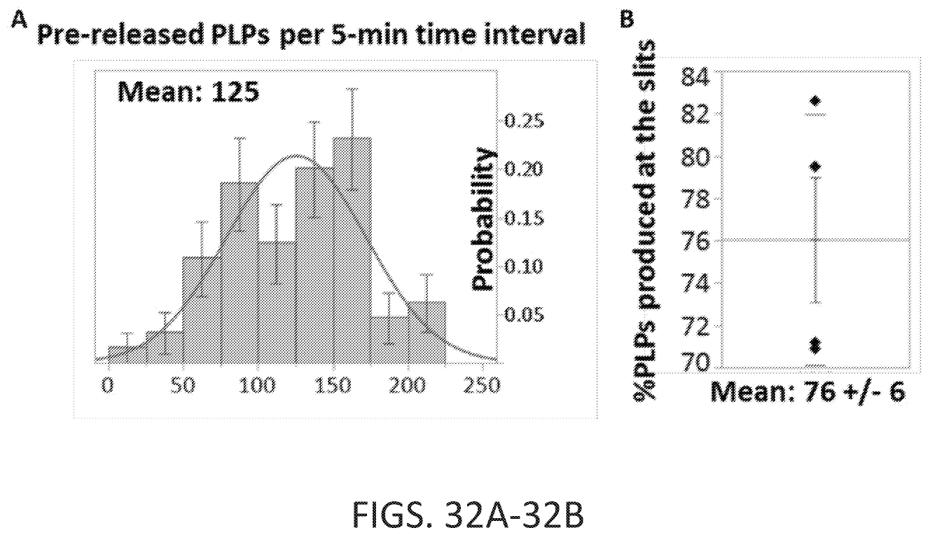

The effluent most likely contained a combination of pre-released particles (present in the Mk suspension introduced into the system) and PLPs generated at the slits. The videos using the counting strategy described earlier were analyzed to determine the rate at which pre-released Calcein-stained particles entered and exited the slits. The mean rate was 125 per 5-min time interval (FIG. 32A), which is higher than the mean rate of PLP generation for a combined outer channel flow rate of 5 μL/min (55), but less than half than the mean rate for a combined outer channel flow rate of 0 μL/min (351). By counting flow-through and newly produced PLPs in the same experiment, it was estimated that approximately 76% of Calcein-stained PLPs were generated by the slits in the reactors with no flow in the outer channels (FIG. 32B). Therefore, the Calcein⁺ PLPs characterized in the effluent were largely generated by Mks trapped at the slits.

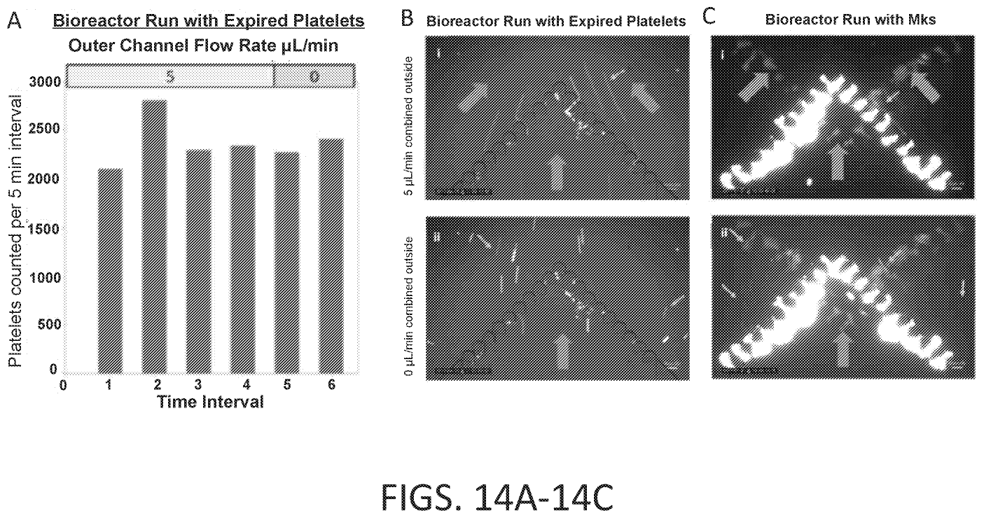

To verify the PLP counting process, expired blood platelets, stained with Calcein, were introduced into a cell-free bioreactor. During a 30-min perfusion, platelets were counted per 5-min time interval for outer channel combined flow rates of 5 μL/min or 0 μL/min (FIG. 14A). The number of platelets counted per time interval was about the same for either condition, as expected, since platelets would only enter via the center channel. Additionally, images of expired platelets flowing through the USRB-7 μm are provided (FIG. 14B) to compare them to the PLPs released from trapped Mks to further support the count strategy (FIG. 14C).

### Example 6

### CFD Analysis of Changes to the Outer Channel Flow Rate

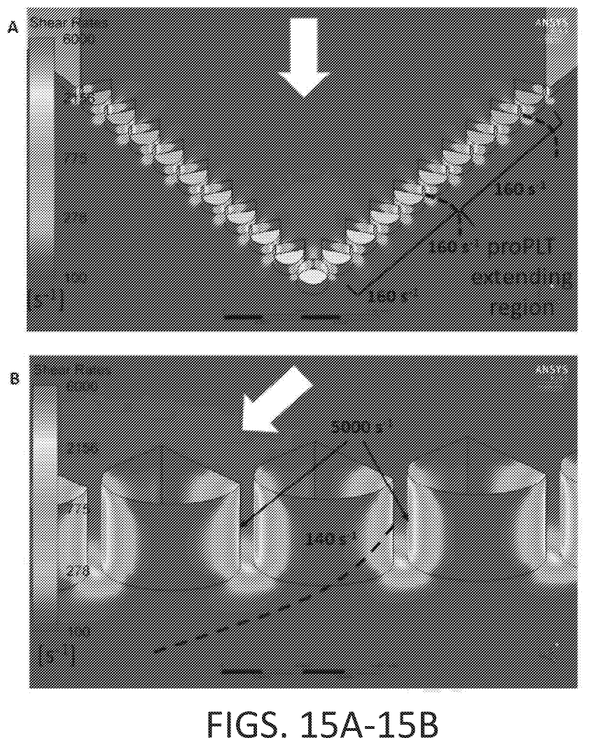

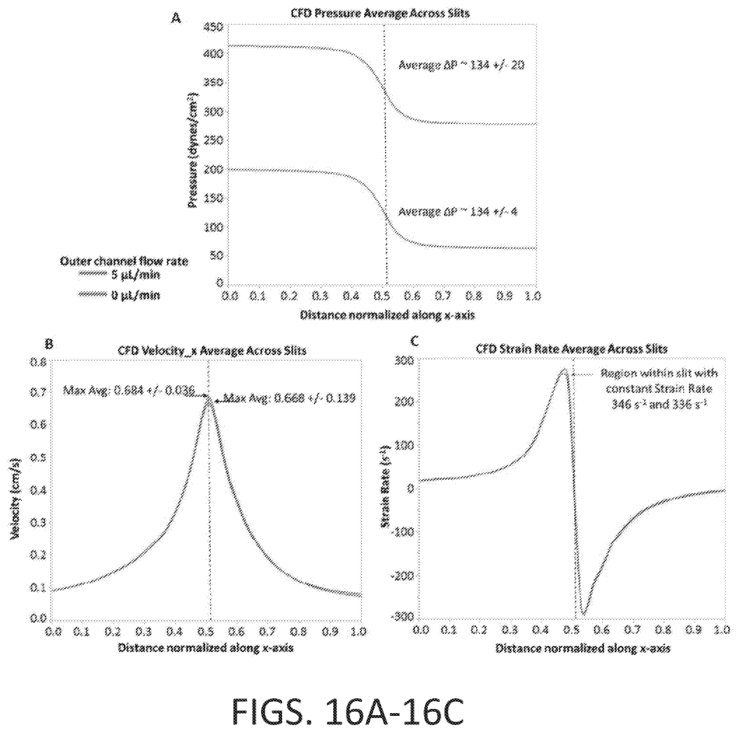

CFD was used to evaluate what environmental factors could explain the differences in Mk behavior at 5 μL/min vs. 0 μL/min flow rates in the outer channels, while keeping the center channel flow rate constant at 1.5 μL/min. Wall shear rate, pressure, velocity, strain rate, and the structure of the flow patterns were the primary factors of interest. Simulations with the outer flow rate of 0 μL/min did not show changes to the wall shear rates within the slits of the bioreactor (FIG. 15 vs. FIG. 5). Next, the predicted pressure and velocity profiles across the slits at the center height of the bioreactor, z=20 μm (FIG. 33A) was investigated using CFD. The average CFD pressure drop across the slits was similar for the two outer channel flow rate conditions, but at 5 μL/min the variability between slits was greater (FIG. 16A and FIG. 33B). The outside flow likely imparts some back pressure in the center channel flow, evident by the higher relative pressures shown in FIG. 16A. Stopping the outside flow potentially reduced the pressure downstream of the slits and may allow Mks to release more PLPs. The velocity profile across the slits is also more variable when the combined outside channel flow rate is 5 μL/min vs. 0 μL/min, but the average velocity profiles were similar (FIG. 16B and FIGS. 33C-33D).

Next, the strain rate (rate of deformation) within the bioreactor slits was examined. Strain rates represent extensional flow that is created due to a velocity gradient in the direction of flow. The CFD outputs of the bioreactor of the present disclosure showed an increase in velocity along the slits, due to the hyperbolic like-converging region (FIG. 16B). In CFD, the strain rate can be extracted from the velocity gradient tensor output as dVx/dx. Plotting the average strain rate across the slits did not show any large differences for combined outer channel flow rates of 5 μL/min or 0 μL/min (FIG. 16C). The maximum strain rates predicted are 336 s⁻¹ for 5 μL/min and 346 s⁻¹ for 0 μL/min outside combined flow rate. Based on this analysis, though there were no differences in the strain rates, extensional flow conditions were observed within these slits.

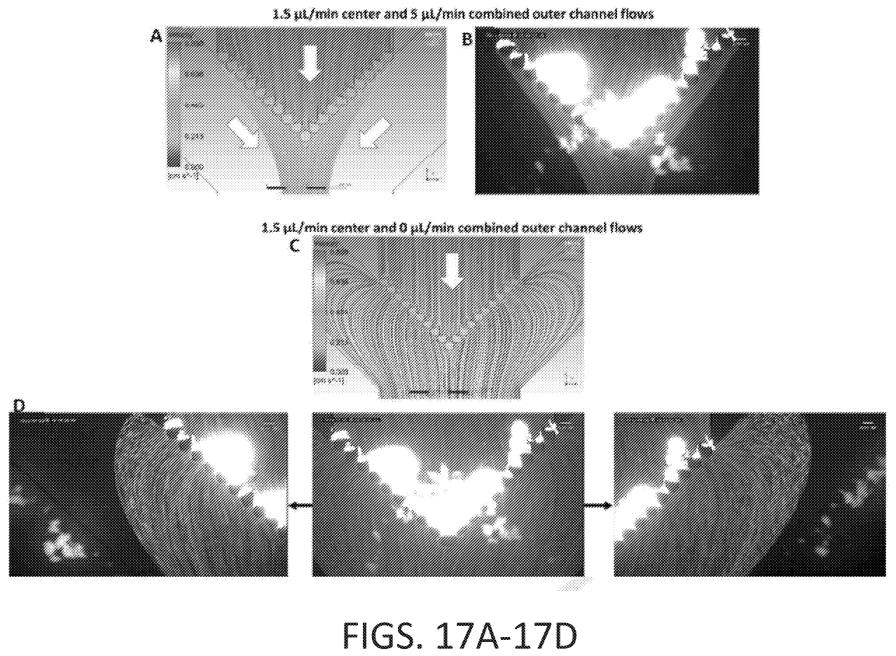

The structure of the flow patterns was assessed using the CFD streamlines, as well as 1-μm fluorescent beads to map the experimental streamlines. There is significant correlation between the predicted and experimental streamlines under the two different outside flow conditions (FIG. 17). The flow patterns are very different at the two flow rates. For the 5 μL/min combined outer channel flow rate, the streamlines are compressed toward the center of the reactor along the posts (FIGS. 17A-17B). On the other hand, with no outer channel flow, the streamlines are not compressed, but rather expand downstream of the slits (FIGS. 17C-17D). Furthermore, overlaying images from Mk experiments with the CFD streamlines demonstrates how the flow structure influences the behavior of the Mks (FIG. 18). This is further supported by the observation of switching the flow from 5 μL/min to 0 μL/min shown in Video S4. Also, the velocity vectors past the slits show that the flows through individual slits seem to interact with each other when the outside flow is at 5 μL/min, whereas a more isolated slit environment is generated with no outside flow (FIG. 19A vs. FIG. 19B).

### Example 7

### Uniform Shear Rate Bioreactor With 5 μm Slits

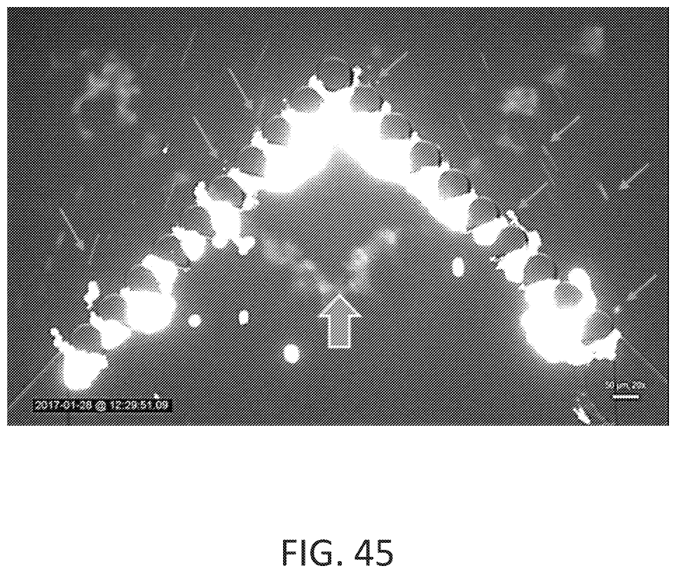

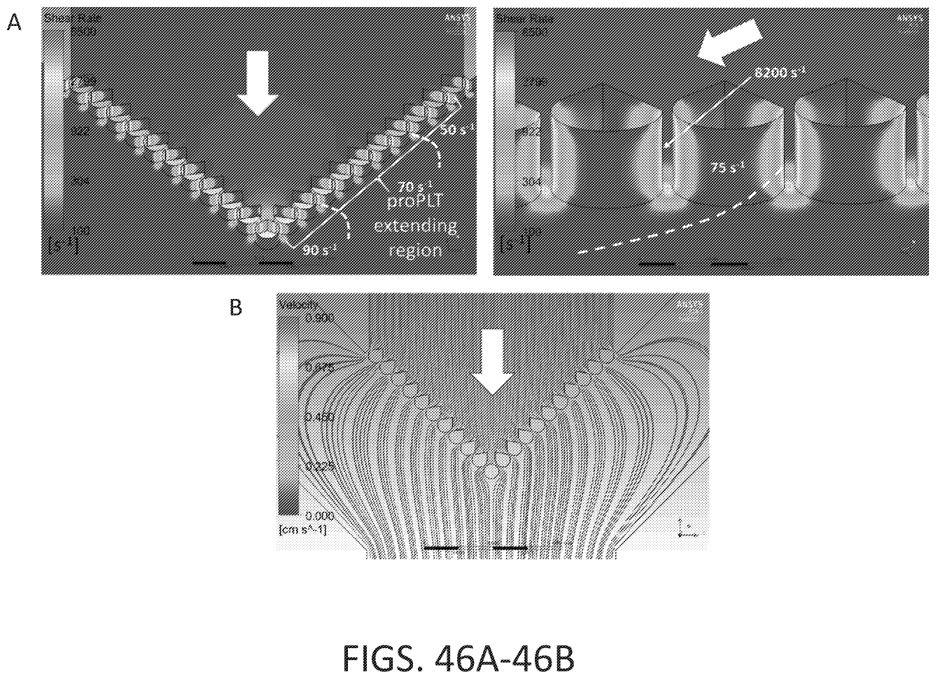

Previous studies have investigated platelet generation from mobilized-peripheral blood (mPB) Mks (e.g., Martinez, A F et al. Biotechnology Progress 2017; 33:1614-1629). Mks can also be derived from cord-blood stem and progenitor cells (CB). These CB-Mks are known to be lower ploidy and smaller in size compared to mPB-Mks. In some embodiments, the USRB-7 μm with CB-Mks showed that though cells can get captured, the overall efficiency was lower due to smaller size as these cells more easily squeezed through the slits. Images depicting the USRB-7 μm loaded with CB-Mks are shown in FIG. 45. These captured CB-Mks at the slits in the presence of shear forces within the bioreactor can make proplatelets and rapidly release individual platelet-like-particles (PLPs) (blue arrows in FIG. 45).

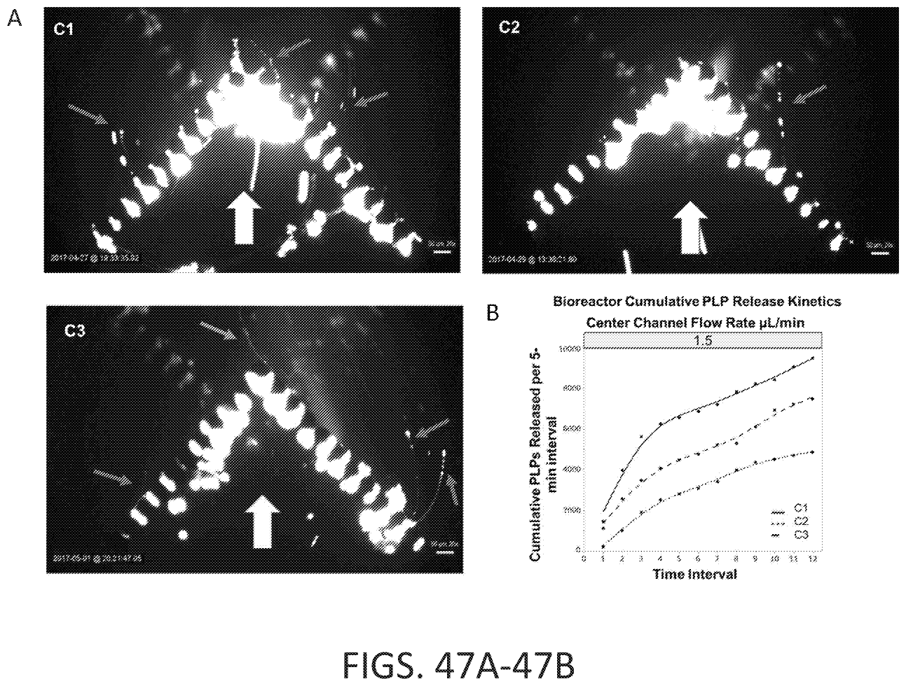

To improve the retention of CB-Mks at the slits, the slits were narrowed to 5 μm and termed “USRB-5 μm,” which retains the same slit geometry and design as the USRB-7 μm described above. This system exposed Mks to a uniform environment with max shear rate at the slits of 8200 s^{'1} and shear on proPLTs at 50-90 s⁻¹ (FIG. 2). The bioreactor was fabricated and experimentally set-up in the same way as the USRB-7 μm (see, e.g., Martinez et al. 2017). Selected CB-derived Mks from three different cultures (C1, C2, or C3), were introduced into USRB-5 μm and the PLP kinetics analyzed over an hour of perfusion. Mks from all culture conditions were observed making proPLTs (FIG. 47A) and rapidly releasing dozens of individual PLPs. As described herein, PLP release kinetics of the system were analyzed through video analysis and it was determined that C1 Mks were more productive within the USRB-5μm, followed by C3 and C2 Mks (FIG. 47B), especially at the beginning of the perfusion run where the rate of PLP releases is higher in C1. Overall, the USRB-5 μm improved the capture and retention of CB-Mks at the slits.

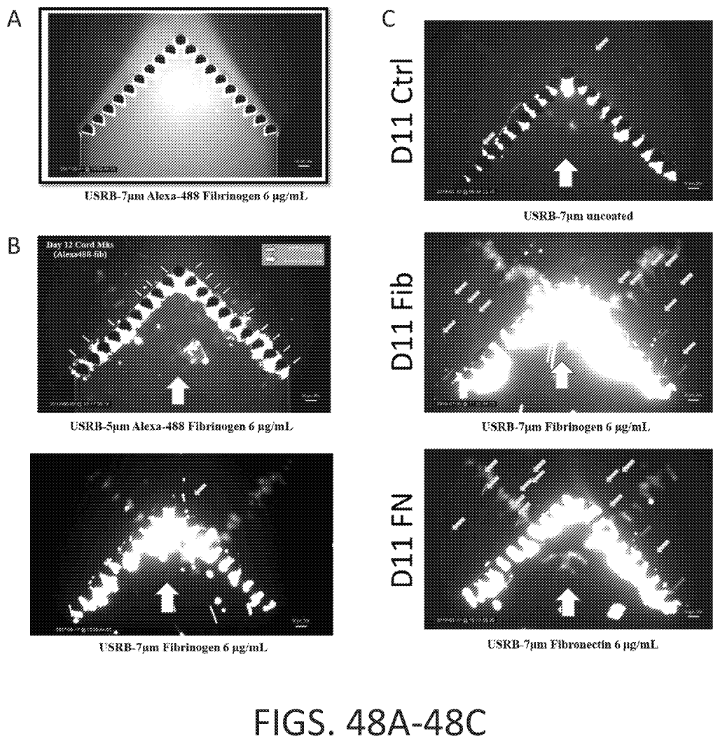

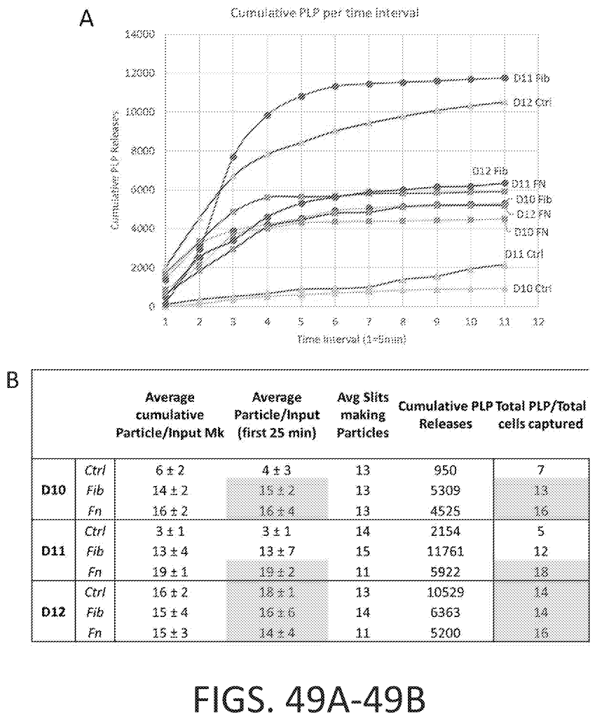

The Mk bone marrow niche is a complicated environment to replicate. Besides cell-cell and cell-extracellular matrix interactions, several abundant proteins exist within this environment, of which fibrinogen and fibronectin had some observed effect on platelet production, among others. Both of these proteins have a positive impact on in vitro proplatelet formation. Due to the design and materials of the USRB-7 μm and USRB-5 μm bioreactors, coatings were introduced into the system to then evaluate Mk-protein interaction in the presence of uniform shear forces to potentially elucidate additional key effects on platelet production. Using a fluorophore-tagged fibrinogen protein, results showed that the USRB systems were uniformly coated across the slits (FIG. 48A). Additionally, mPB-Mks or CB-Mks were introduced into the fibrinogen coated environment to demonstrate that Mks can produce proplatelets and PLPs (FIG. 48B). Mks appeared stickier and were retained more efficiently at the slits when fibrinogen or fibronectin was on the surface (FIG. 48C). Using video analysis methods, the cumulative PLP production over different bioreactor runs across multiple days of culture was plotted. These data demonstrated that the protein coatings could generate more proplatelet and PLP generation earlier in culture compared to uncoated conditions (FIG. 49). The difference in PLP production between protein coated conditions and the uncoated control demonstrated that there is some interaction occurring between fibrinogen and fibronectin to some degree with the Mks thus far studied.

The data presented herein show how the USRB systems can be used to characterize Mk behavior within well-defined microfluidic environments. Changing the slit size to allow better capture facilitated more efficient investigations of CB-Mks. Additionally, incorporating other aspects of the bone marrow microenvironment into the USRBs will allow further analysis platelet generation from Mks.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. A microfluidic proplatelet (proPLT) and platelet-like particle (PLP) production chamber device comprising:
a plurality of slit channels comprising one or more proPLT/PLP production slits configured to expose a megakaryocyte to a uniform shear profile to facilitate the production of proPLTs/PLPs;
wherein the plurality of slit channels is distal to and in fluid communication with a central flow channel.

2. The device of claim 1, wherein the one or more proPLT/PLP production slits are from about 3 μm to about 10 μm wide.

3-4. (canceled)

5. The device of claim 1, wherein the uniform shear profile produces a maximum shear rate at the one or more proPLT/PLP production slits from about 5000 s⁻¹ to about 10,000 s⁻¹.

6. The device of claim 1, wherein the uniform shear profile produces a maximum shear rate at the one or more proPLT/PLP production slits from about 5000 s⁻¹ to about 8,000 s⁻¹.

7. The device of claim 1, wherein the uniform shear profile produces a maximum shear rate at the one or more proPLT/PLP production slits of about 5000 s⁻¹, and wherein the one or more proPLT/PLP production slits are about 7 μm wide.

8. The device of claim 1, wherein the uniform shear profile produces a maximum shear rate at the one or more proPLT/PLP production slits of about 8,200 s⁻¹, and wherein the one or more proPLT/PLP production slits are about 5 μm wide.

9. The device claim 1, wherein the uniform shear profile produces a shear rate on the proPLTs/PLPs ranging from about 50 s⁻¹ to about 500 s⁻¹.

10. The device of claim 1, wherein the uniform shear profile produces a shear rate on the proPLTs/PLPs ranging from about 250 s⁻¹ to about 350 s⁻¹.

11. (canceled)

12. The device of claim 1, wherein the uniform shear profile produces a shear rate on the proPLTs/PLPs ranging from about 250 s⁻¹ to about 350 s⁻¹, and wherein the one or more proPLT/PLP production slits are about 7 μm wide.

13. The device of claim 1, wherein the uniform shear profile produces a shear rate on the proPLTs/PLPs ranging from about 50 s⁻¹ to about 100 s⁻¹, and wherein the one or more proPLT/PLP production slits are about 5 μm wide.

14. The device of claim 1, wherein fluid flows in a single direction from the central flow channel to the plurality of slit channels.

15. The device of claim 14, wherein the single direction fluid flow produces at least a 5-fold increase in proPLT/PLP production.

16. (canceled)

17. A microfluidic bioreactor device for the production of proplatelets (proPLTs) and platelet-like particles (PLPs), the device comprising:
a megakaryocyte loading reservoir coupled to a central flow channel;
a branching flow channel in fluid communication with the central flow channel; and
a proPLT/PLP production chamber comprising a plurality of slit channels distal to and in fluid communication with the branching flow channel, wherein the plurality of slit channels comprise one or more proPLT/PLP production slits configured to expose a megakaryocyte to a uniform shear profile to facilitate the production of proPLTs/PLPs.

18. The device of claim 17, wherein the one or more proPLT/PLP production slits are from about 5 μm to about 7 μm wide.

19. The device of claim 17, wherein the uniform shear profile produces a maximum shear rate at the one or more proPLT/PLP production slits of about 5000 s⁻¹, and wherein the one or more proPLT/PLP production slits are about 7 μm wide.

20. The device of claim 17, wherein the uniform shear profile produces a maximum shear rate at the one or more proPLT/PLP production slits of about 8,200 s⁻¹, and wherein the one or more proPLT/PLP production slits are about 5 μm wide.

21. The device of claim 17, wherein the uniform shear profile produces a shear rate on the proPLTs/PLPs ranging from about 250 s⁻¹ to about 350 s⁻¹, and wherein the one or more proPLT/PLP production slits are about 7 μm wide.

22. The device of claim 17, wherein the uniform shear profile produces a shear rate on the proPLTs/PLPs ranging from about 50 s⁻¹ to about 100 s⁻¹, and wherein the one or more proPLT/PLP production slits are about 5 μm wide.

23. The device of claim 17, wherein fluid flows in a single direction from the central flow channel to the plurality of slit channels.

24. A microfluidic bioreactor system for the production of proplatelets (proPLTs) and platelet-like particles (PLPs), the system comprising:
a megakaryocyte loading reservoir coupled to a central flow channel;
a branching flow channel in fluid communication with the central flow channel; and
a proPLT/PLP production chamber comprising a plurality of slit channels distal to and in fluid communication with the branching flow channel, wherein the plurality of slit channels comprise one or more proPLT/PLP production slits configured to expose a megakaryocyte to a uniform shear profile to facilitate the production of proPLTs/PLPs.

25-26. (canceled)
